(19) United States
(12) Patent Application Publication
(10) Pub. No.: US 2007/0053878 A1
(43) Pub. Date: Mar. 8, 2007

(54) Sars

(76) Inventors: Bartholomeus Leonardus Haagmans, Abcoude (NL); Thijs Kuiken, Gouda (NL); Ronaldus Adrianus Maria Fouchier, Rotterdam (NL); Albertus Dominicus Marcellinus Erasmu Osterhaus, Bunnik (NL)

Correspondence Address: HOFFMANN & BARON, LLP, 6900 JERICHO TURNPIKE, SYOSSET, NY, 11791, US

(21) Appl. No.: 10/552,755

(22) PCT Filed: Apr. 8, 2004
(86) PCT No.: PCT/NL04/00229

§ 371 Date: Jun. 26, 2006

(30) Foreign Application Priority Data
Apr. 8, 2003 (EP) 03076037.5
Apr. 14, 2003 (EP) 03076110.0
Jul. 18, 2003 (EP) 03077307.1

(51) Int. Cl. C12Q 1/70; C07H 21/04; A61K 38/21; C07K 14/165

(52) U.S. Cl. 424/85.7; 435/5; 435/235.1; 530/388.3; 530/350; 536/23.72; 435/325

(57) ABSTRACT

The invention relates to the field of virology. The invention provides an isolated essentially mammalian positive-sense single stranded RNA virus (SARS) within the group of coronaviuses and components thereof.

The invention relates to the field of virology, more in particular to a new coronavirus. In particular sequences encoding (parts of) viral proteins are provided. Further, the invention relates to diagnostic means and methods, prophylactic means and methods and therapeutic means and methods to be employed in the diagnosis, prevention and/or treatment of disease, in particular of respiratory disease (atypical pneumonia), in particular of mammals, more in particular in humans. In another embodiment the invention relates to the use of interferon, preferably pegylated interferon for the prophylactic or therapeutic treatment of animals, preferably vertebrates, more preferably birds or mammals, especially human, apes or rodents, infected with a coronavirus, more specifically an animal, preferably human infected with a SARS associated coronavirus (SARS-CoV).

Recently, a new virus has caused a global health risk because of its pathogenic effects in man combined with a relatively easy droplet transmission. The virus first was seen in the Chinese province Guangdong, was spread to Hong Kong in February 2003, and within two months it has been able to spread to several countries all over the world where it has caused 78 deaths out of 2300 people infected (New Scientist Online News 13:25 Apr. 2, 2003). The virus has been named SARS (Severe Acute Respiratory Syndrome) virus and causes a respiratory illness (atypical pneumonia) in man. This illness usually begins with a fever, sometimes associated with chills or other symptoms, including headache, rash, diarrhea, a general feeling of discomfort (malaise) and body aches. Some people also experience mild respiratory syndromes at the outset.

After 2 to 7 days, SARS patients may develop a dry, nonproductive cough that might be accompanied or progress to the point where insuffiecient oxygen is getting to the blood, visible as shortness of breath. In 10% to 20% of the cases, patients will require mechanical ventilation, and eventually the disease can lead to the death of the patient. Hospital personnel, children, elderly and people having an underlying condition such as diabetes or heart disease, or a weakened immune system, form the highest risk group. Co-infection with other pathogens seems to occur frequently, especially with opportunistic pathogenic microorganisms such as human metapneumovirus (hMPV), Chlamydia, etcetera.

The incubation time for the virus is typically 2-7 days and the disease is transmitted by people sich with SARS coughing or sneezing droplets in the air.

As for yet it is not known if there is a cure for the disease. Several antiviral therapies have been applied, but with various results.

Also, for being able to prevent spread of the disease, it is of great importance to be able to recognise the disease in an early stage. Only then sufficient measures can be taken to isolate patients and initiate quarantaine precautions. At this moment there is not yet a diagnostic tool in place.

Thus, there is great need in developing diagnostic tools and therapies for this disease.

The invention provides the nucleotide sequence of an isolated essentially mammalian positive-sense single stranded RNA virus belonging to the Coronaviruses, which is the causative factor for SARS. From a phylogenetic analysis of the sequences of the virus (FIG. 1) it appears that the virus is an intermediate between the group formed by TGEV (transmissable gastroenetritis virus), PEDV (porcine epidemic diarrhea virus) and 229E (human coronavirus 229E) at one side, the group formed by BoCo (bovine coronavirus) and MHV (murine hepatitis virus) at an other side, and the AIBV (avian infectious bronchitis virus) on yet another side. In general, bovine coronavirus seems to be the closest relative (at least for the viral replicase protein).

Although phylogenetic analyses provide a convenient method of identifying a virus as a SARS virus several other possibly more straightforward albeit somewhat more coarse methods for identifying said virus or viral proteins or nucleic acids from said virus are herein also provided. As a rule of thumb a SARS virus can be identified by the percentages of homology of the virus, proteins or nucleic acids to be identified in comparison with viral proteins or nucleic acids identified herein by sequence. It is generally known that virus species, especially RNA virus species, often constitute a quasi species wherein a cluster of said viruses displays heterogeneity among its members. Thus it is expected that each isolate may have a somewhat different percentage relationship with the sequences of the isolate as provided herein.

When one wishes to compare a virus isolate with the sequences as listed in FIG. 2, the invention provides an isolated essentially mammalian positive-sense single stranded RNA virus (SARS) belonging to the Coronaviruses and identifiable as phylogenetically corresponding thereto by determining a nucleic acid sequence of said virus and determining that said nucleic acid sequence has a percentage nucleic acid identity to the sequences as listed higher than the percentages identified herein for the nucleic acids as identified herein below in comparison with BoCo, AIPV and PEDV. Likewise, an isolated essentially mammalian positive-sense single stranded RNA virus (SARS) belonging to the Coronaviruses and identifiable as phylogenetically corresponding thereto by determining an amino acid sequence of said virus and determining that said amino acid sequence has a percentage amino acid homology to the sequences as listed which is essentially higher than the percentages provided herein in comparison with BoCo, AIPV and PEDV.

With the provision of the sequence information of this SARS virus, the invention provides diagnostic means and methods, prophylactic means and methods and therapeutic means and methods to be employed in the diagnosis, prevention and/or treatment of disease, in particular of respiratory disease (atypical pneumonia), in particular of mammals, more in particular in humans. In virology, it is most advisory that diagnosis, prophylaxis and/or treatment of a specific viral infection is performed with reagents that are most specific for said specific virus causing said infection. In this case this means that it is preferred that said diagnosis, prophylaxis and/or treatment of a SARS virus infection is performed with reagents that are most specific for SARS virus. This by no means however excludes the possibility that less specific, but sufficiently cross-reactive reagents are used instead, for example because they are more easily available and sufficiently address the task at hand. The invention for example provides a method for virologically diagnosing a SARS infection of an animal, in particular of a mammal, more in particular of a human being, comprising determining in a sample of said animal the presence of a viral isolate or component thereof by reacting said sample with a SARS specific nucleic acid or antibody according to the invention, and a method for serologically diagnosing a SARS infection of a mammal comprising determining in a sample of said mammal the presence of an antibody specifically directed against a SARS virus or component thereof by reacting said sample with a SARS virus-specific proteinaceous molecule or fragment thereof or an antigen according to the invention. The invention also provides a diagnostic kit for diagnosing a SARS infection comprising a SARS virus, a SARS virus-specific nucleic acid, proteinaceous molecule or fragment thereof, antigen and/or an antibody according to the invention, and preferably a means for detecting said SARS virus, SARS virus-specific nucleic acid, proteinaceous molecule or fragment thereof, antigen and/or an antibody, said means for example comprising an excitable group such as a fluorophore or enzymatic detection system used in the art (examples of suitable diagnostic kit format comprise IF, ELISA, neutralization assay, RT-PCR assay). To determine whether an as yet unidentified virus component or synthetic analogue thereof such as nucleic acid, proteinaceous molecule or fragment thereof can be identified as SARS-virus-specific, it suffices to analyse the nucleic acid or amino acid sequence of said component, for example for a stretch of said nucleic acid or amino acid, preferably of at least 10, more preferably at least 25, more preferably at least 40 nucleotides or amino acids (respectively), by sequence homology comparison with the provided SARS viral sequences and with known non-SARS viral sequences (BoCo is preferably used) using for example phylogenetic analyses as provided herein. Depending on the degree of relationship with said SARS or non-SARS viral sequences, the component or synthetic analogue can be identified.

The invention thus provides the nucleotide sequence of a novel etiological agent, an isolated essentially mammalian positive-sense single stranded RNA virus (herein also called SARS virus) belonging to the Coronaviridae family, and SARS virus-specific components or synthetic analogues thereof. Coronaviruses were first isolated from chickens in 1937, while the first human coronavirus was propagated in vitro by Tyrell and Bonoe in 1965. There are now about 13 species in this family, which infect cattle, pigs, rodents, cats, dogs, birds and man. Coronavirus particles are irregularly shaped, about 60-220 nm in diameter, with an outer envelope bearing distinctive, ‘club-shaped’ peplomers (about 20 nm long and 10 nm wide at the distal end). This ‘crown-like’ appearance give the family its name. The envelope carries two glycoproteins: S, the spike glycoprotein which is involved in cell fusion and is a major antigen, and M, the membrane glycoprotein, which is involved in budding and envelope formation. The genome is associated with a basic phosphoprotein, designated N. The genome of coronaviruses, a single stranded positive-sense RNA strand, is typically 27-31 Kb long and contains a 5′ methylated cap and a 3′ poly-A tail, by which it can directly function as an mRNA in the infected cell. Initially the 5′ ORF 1 (about 20 Kb) is translated to produce a viral polymerase, which then produces a full length negative sense strand. This is used as a template to produce mRNA as a ‘nested set’ of transcripts, all with identical 5′ non-translated leader sequence of 72 nucleotides and coincident 3′ polyadenylated ends. Each mRNA thus produced is monocistronic, the genes at the 5′ end being translated from the longest mRNA and so on. These unusual cytoplasmic structures are produced not by splicing, but by the polymerase during transcription. Between each of the genes there is a repeated intergenic sequence—AACUAAAC—which interacts with the transcriptase plus cellular factors to splice the leader sequence onto the start of each ORF. In some coronaviruses there are about 8 ORFs, coding for the proteins mentioned above, but also for a heamagglutenin esterase (HE), and several other non-structural proteins. Newly isolated viruses are phylogenetically corresponding to and thus taxonomically corresponding to SARS virus when comprising a gene order and/or amino acid sequence and/or nucleotide sequence sufficiently similar to our prototypic SARS virus. The highest amino acid sequence homology, between SARS virus and any of the known other viruses of the same family to date (BoCo or Mouse Hapatitis Virus) is for parts of the polymerase protein 18-61% (the % homology, and the virus to which the homology is depend on the region of the polymerase that is examined), as can be deduced when comparing the sequences given in FIG. 2 with sequences of other viruses, in particular of BoCo and Mouse Hapatitis Virus. Individual proteins or whole virus isolates with, respectively, higher homology than these mentioned maximum values are considered phylogenetically corresponding and thus taxonomically corresponding to SARS virus, and generally will be encoded by a nucleic acid sequence structurally corresponding with a sequence as shown in FIG. 2. Herewith the invention provides a virus phylogenetically corresponding to the isolated virus of which the sequences are depicted in FIG. 2. It should be noted that, similar to other viruses, a certain degree of variation can be expected to be found between SARS-viruses isolated from different sources. Also, the viral sequence of the SARS virus or an an isolated SARS virus gene as provided herein for example shows less than 95%, preferably less than 90%, more preferably less than 80%, more preferably less than 70% and most preferably less than 65% nucleotide sequence homology or less than 95%, preferably less than 90%, more preferably less than 80%, more preferably less than 70% and most preferably less than 65% amino acid sequence homology with the respective nucleotide or amino acid sequence of the bovine coronavirus or the murine hepatitis virus as for example can be found in Genbank (for example in accession number NC_{—}002306 (BoCo) or NC_{—}002645 (MHV).

Sequence divergence of SARS strains around the world may be somewhat higher, in analogy with other coronaviruses.

A fair number of virus isolates have been isolated during the priority year of the present application, and it has been found that these viruses share the homology indicated above. The sequences of these viruses can be found in GenBank accession no. AY274119 (see FIG. 10) or AY278741 or AY338175 or AY338174 or AY322199 or AY 322198 or AY322197 or AH013000 or AY322208 or AY322207 AY 322206 or AY322205 or AH012999 and and/or sequences depicted in http://www.ncbi.nlm.nih.gov/Taxonomy/Browser/wwwtax.cgi?mode=Undef&id=227859&lvl=3&keep=1&srchmode=1&unlock. Herewith the invention encompasses a virus phylogenetically corresponding to the isolated virus of which the sequences are depicted in FIG. 2 and/or for example the GenBank accession no. AY274119 or AY278741 or AY338175 or AY338174 or AY322199 or AY 322198 or AY322197 or AH013000 or AY322208 or AY322207 AY 322206 or AY322205 or AH012999 and and/or sequences depicted in http://www.ncbi.nlm.nih.gov/Taxonomy/Browser/wwwtax.cgi?mode=Undef&id=227859&lvl=3&keep=1&srchmode=1&unlock.

The term “nucleotide sequence homology” as used herein denotes the presence of homology between two (poly)nucleotides. Polynucleotides have “homologous” sequences if the sequence of nucleotides in the two sequences is the same when aligned for maximum correspondence. Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence homology” for polynucleotides, such as 50, 60, 70, 80, 90, 95, 98, 99 or 100 percent sequence homology may be determined by comparing two optimally aligned sequences over a comparison window, wherein the portion of the polynucleotide sequence in the comparison window may include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology. Optimal alignment of sequences for comparison may be conducted by computerized implementations of known algorithms, or by inspection. Readily available sequence comparison and multiple sequence alignment algorithms are, respectively, the Basic Local Alignment Search Tool (BLAST) (Altschul, S. F. et al. 1990. J. Mol. Biol. 215:403; Altschul, S. F. et al. 1997. Nucleic Acid Res. 25:3389-3402) and ClustalW programs both available on the internet. Other suitable programs include GAP, BESTFIT and FASTA in the Wisconsin Genetics Software Package (Genetics Computer Group (GCG), Madison, Wis., USA).

As used herein, “substantially complementary” means that two nucleic acid sequences have at least about 65%, preferably about 70%, more preferably about 80%, even more preferably 90%, and most preferably about 98%, sequence complementarity to each other. This means that the primers and probes must exhibit sufficient complementarity to their template and target nucleic acid, respectively, to hybridise under stringent conditions. Therefore, the primer sequences as disclosed in this specification need not reflect the exact sequence of the binding region on the template and degenerate primers can be used. A substantially complementary primer sequence is one that has sufficient sequence complementarity to the amplification template to result in primer binding and second-strand synthesis.

The term “hybrid” refers to a double-stranded nucleic acid molecule, or duplex, formed by hydrogen bonding between complementary nucleotides. The terms “hybridise” or “anneal” refer to the process by which single strands of nucleic acid sequences form double-helical segments through hydrogen bonding between complementary nucleotides.

The term “oligonucleotide” refers to a short sequence of nucleotide monomers (usually 6 to 100 nucleotides) joined by phosphorous linkages (e.g., phosphodiester, alkyl and aryl-phosphate, phosphorothioate), or non-phosphorous linkages (e.g., peptide, sulfamate and others). An oligonucleotide may contain modified nucleotides having modified bases (e.g., 5-methyl cytosine) and modified sugar groups (e.g., 2′-O-methyl ribosyl 2′-O-methoxyethyl ribosyl, 2′-fluoro ribosyl, 2′-amino ribosyl, and the like). Oligonucleotides may be naturally-occurring or synthetic molecules of double- and single-stranded DNA and double- and single-stranded RNA with circular, branched or linear shapes and optionally including domains capable of forming stable secondary structures (e.g., stem-and-loop and loop-stem-loop structures).

The term “primer” as used herein refers to an oligonucleotide which is capable of annealing to the amplification target allowing a DNA polymerase to attach thereby serving as a point of initiation of DNA synthesis when placed under conditions in which synthesis of primer extension product which is complementary to a nucleic acid strand is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxy ribonucleotide. The primer must be sufficiently long to prime the synthesis of extension products in the presence of the agent for polymerization. The exact lengths of the primers will depend on many factors, including temperature and source of primer. A “pair of bi-directional primers” as used herein refers to one forward and one reverse primer as commonly used in the art of DNA amplification such as in PCR amplification.

The term “probe” refers to a single-stranded oligonucleotide sequence that will recognize and form a hydrogen-bonded duplex with a complementary sequence in a target nucleic acid sequence analyte or its cDNA derivative.

The terms “stringency” or “stringent hybridization conditions” refer to hybridization conditions that affect the stability of hybrids, e.g., temperature, salt concentration, pH, formamide concentration and the like. These conditions are empirically optimised to maximize specific binding and minimize non-specific binding of primer or probe to its target nucleic acid sequence. The terms as used include reference to conditions under which a probe or primer will hybridise to its target sequence, to a detectably greater degree than other sequences (e.g. at least 2-fold over background). Stringent conditions are sequence dependent and will be different in different circumstances. Longer sequences hybridise specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridises to a perfectly matched probe or primer. Typically, stringent conditions will be those in which the salt concentration is less than about 1.0 M Na+ ion, typically about 0.01 to 1.0 M Na+ ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1 M NaCl, 1% SDS at 37° C., and a wash in 0.1×SSC at 60° C. Hybridization procedures are well known in the art and are described in e.g. Ausubel et al, Current Protocols in Molecular Biology, John Wiley & Sons Inc., 1994.

The term “antibody” includes reference to antigen binding forms of antibodies (e. g., Fab, F(ab)2). The term “antibody” frequently refers to a polypeptide substantially encoded by an immunoglobulin gene or immunoglobulin genes, or fragments thereof which specifically bind and recognize an analyte (antigen). However, while various antibody fragments can be defined in terms of the digestion of an intact antibody, one of skill will appreciate that such fragments may be synthesized de novo either chemically or by utilizing recombinant DNA methodology. Thus, the term antibody, as used herein, also includes antibody fragments such as single chain Fv, chimeric antibodies (i. e., comprising constant and variable regions from different species), humanized antibodies (i. e., comprising a complementarity determining region (CDR) from a non-human source) and heteroconjugate antibodies (e. g., bispecific antibodies).

“Interferon” is a term generically comprehending a group of vertebrate glycoproteins and proteins which are known to have various biological activities, such as antiviral, antiproliferative, and immunomodulatory activity at least in the species of animal from which such substances are derived. Interferon refers to a class of small protein and glycoprotein cytokines (15-28 kD) produced by T cells, fibroblasts, and other cells in response to viral infection and other biological and synthetic stimuli. Interferons bind to specific receptors on cell membranes; their effects include inducing enzymes, suppressing cell proliferation, inhibiting viral proliferation, enhancing the phagocytic activity of macrophages, and augmenting the cytotoxic activity of T lymphocytes. Interferons are divided into five major classes (alpha, beta, gamma, tau, and omega) and several subclasses (indicated by Arabic numerals and letters) on the basis of physicochemical properties, cells of origin mode of induction, and antibody reactions.

In short, the invention provides an isolated essentially mammalian positive-sense single stranded RNA virus (SARS) belonging to the Coronaviruses and identifiable as phylogenetically corresponding thereto by determining a nucleic acid sequence of a suitable fragment of the genome of said virus and testing it in phylogenetic tree analyses wherein maximum likelihood trees are generated using 100 bootstraps and 3 jumbles and finding it to be more closely phylogenetically corresponding to a virus isolate having the sequences as depicted in FIG. 2 than it is corresponding to a virus isolate of BoCo (bovine coronavirus, e.g. acc. no. NC_{—}002306 in Genbank), MHV (murine hepatitis virus, e.g. acc. no. NC_{—}002645), AIBV (avian infectious bronchitis virus, e.g. ace. no. NC_{—}001451), PEDV (porcine epidemic diarrhea virus), TGEV (transmissible gastroenteritis virus, e.g. acc. no. NC_{—}003436) or 229E (human coronavirus 229E, e.g. acc. no. NC_{—}003045). All the viral sequences with the GenBank accession numbers mentioned above are believed to be phylogentically cooresponding viruses to the virus of which the sequences are depicted in FIG. 2.

Suitable nucleic acid genome fragments each useful for such phylogenetic tree analyses are for example any of the RAP-PCR fragments EMC-1 to -14 and RDG-1 as disclosed in FIG. 2, leading to the phylogenetic tree analysis as disclosed in FIG. 1.

A suitable open reading frame (ORF) comprises the ORF encoding the viral polymerase (ORF 1a). When an overall amino acid identity of at least 60%, preferably of at least 70%, more preferably of at least 80%, more preferably of at least 90%, most preferably of at least 95% of the analysed polymerase with the polymerase having a sequence comprising the amino acid fragments EMC-1, EMC-2, EMC-3, EMC-4,EMC-5, EMC-13 and/or EMC-14 of FIG. 2 is found, the analysed virus isolate comprises a SARS virus isolate according to the invention.

Another suitable open reading frame (ORF) useful in phylogenetic analyses comprises the ORF encoding the N protein. When an overall amino acid identity of at least 60%, more preferably of at least70%, more preferably of at least 80%, more preferably of at least 90%, most preferably of at least 95% of the analysed N-protein with the N-protein encoded by a sequence comprising the sequence EMC-8 of FIG. 2 is found, the analysed virus isolate comprises a SARS isolate according to the invention.

Another suitable open reading frame (ORF) useful in phylogenetic analyses comprises the ORF encoding the spike protein S. When an overall amino acid identity of at least 60%, more preferably of at least 70%, more preferably of at least 80%, more preferably of at least 90%, most preferably of at least 95% of the analysed S-protein encoded by a sequence comprising the sequence of translation 2 of EMC7 and translation 1 of the RDG 1 sequence of the S-protein as depicted in FIG. 2 is found, the analysed virus isolate comprises a SARS virus isolate according to the invention. The S ORF of the SARS virus seems to be located adjacent to the ORF lab (coding for the viral polymerase), which would discriminate SARS viruses from the bovine coronavirus and the murine hepatitis virus, which have a so-called 2a gene and an HE-gene between the S protein and the viral polymerase.

The invention provides among others an isolated or recombinant nucleic acid or virus-specific functional fragment thereof obtainable from a virus according to the invention. The isolated or recombinant nucleic acids comprises the sequences as given in FIG. 2 or sequences of homologues which are able to hybridise with those under stringent conditions. In particular, the invention provides primers and/or probes suitable for identifying a SARS virus nucleic acid.

Furthermore, the invention provides a vector comprising a nucleic acid according to the invention. To begin with, vectors such as plasmid vectors containing (parts of) the genome of SARS virus, virus vectors containing (parts of) the genome of SARS (for example, but not limited thereto, vaccinia virus, retroviruses, baculovirus), or SARS virus containing (parts of) the genome of other viruse or other pathogens are provided.

Also, the invention provides a host cell comprising a nucleic acid or a vector according to the invention. Plasmid or viral vectors containing the polymerase components of SARS virus are generated in prokaryotic cells for the expression of the components in relevant cell types (bacteria, insect cells, eukaryotic cells). Plasmid or viral vectors containing full-length or partial copies of the SARS virus genome will be generated in prokaryotic cells for the expression of viral nucleic acids in-vitro or in-vivo. The latter vectors may contain other viral sequences for the generation of chimeric viruses or chimeric virus proteins, may lack parts of the viral genome for the generation of replication defective virus, and may contain mutations, deletions or insertions for the generation of attenuated viruses.

Infectious copies of SARS virus (being wild type, attenuated, replication-defective or chimeric) can be produced upon co-expression of the polymerase components according to the state-of-the-art technologies described above.

In addition, eukaryotic cells, transiently or stably expressing one or more full-length or partial SARS virus proteins can be used. Such cells can be made by transfection (proteins or nucleic acid vectors), infection (viral vectors) or transduction (viral vectors) and may be useful for complementation of mentioned wild type, attenuated, replication-defective or chimeric viruses.

A chimeric virus may be of particular use for the generation of recombinant vaccines protecting against two or more viruses. For example, it can be envisaged that a SARS virus vector expressing one or more proteins of a human metapneumovirus or a human metapneumovirus vector expressing one or more proteins of SARS virus will protect individuals vaccinated with such vector against both virus infections. Such a specific chimeric virus is particularly useful in the invention because it is suspected that co-infection of, for instance, human metapneumovirus frequently occurs in SARS virus infected patients. Attenuated and replication-defective viruses may be of use for vaccination purposes with live vaccines as has been suggested for other viruses. Recently, Subbarao, K et al., J. Virol. 78(7), 3572-3577, 2004) demonstrated that mice are protected as a result from a previous immunisation with whole viruses.

In a preferred embodiment, the invention provides a proteinaceous molecule or coronavirus-specific viral protein or functional fragment thereof encoded by a nucleic acid according to the invention. Useful proteinaceous molecules are for example derived from any of the genes or genomic fragments derivable from a virus according to the invention. Such molecules, or antigenic fragments thereof, as provided herein, are for example useful in diagnostic methods or kits and in pharmaceutical compositions such as sub-unit vaccines and inhibitory peptides. Particularly useful are the viral polymerase protein, the spike protein, the nucleocapsid or antigenic fragments thereof for inclusion as antigen or subunit immunogen, but inactivated whole virus can also be used. Particulary useful are also those proteinaceous substances that are encoded by recombinant nucleic acid fragments that are identified for phylogenetic analyses, of course preferred are those that are within the preferred bounds and metes of ORFs useful in phylogenetic analyses, in particular for eliciting SARS virus specific antibodies, whether in vivo (e.g. for protective puposes or for providing diagnostic antibodies) or in vitro (e.g. by phage display technology or another technique useful for generating synthetic antibodies).

Also provided herein are antibodies, be it natural polyclonal or monoclonal, or synthetic (e.g. (phage) library-derived binding molecules) antibodies that specifically react with an antigen comprising a proteinaceous molecule or SARS virus-specific functional fragment thereof according to the invention. A person skilled in the art will be able to develop (monoclonal) antibodies using isolated virus material and/or recombinantly expressed viral proteins. Sui et al. (Proc. Natl. Acad. Sci. 101(8), 2536-2541, 2004) have transiently expressed fragments of the spike protein and found several antibodies through phage display methods. One of these antibodies was shown to be directed to the N-terminal 261-672 amino acids of the S (spike) protein (which would be corresponding to the sequence of translation 2 of EMC7 and translation 1 of the RDG 1 sequence of the S-protein as depicted in FIG. 2) and this antibody was also demonstrated to have neutralising properties, indicating that it may be a candidate for succesfull vaccines. Also Subbarao et al. (supra) showed that serum from mice that had been infected with SARS virus was able to block infectivity of 100 TCID₅₀ of SARS virus in Vero cell monolayers, due to the presence of neutralising antibodies.

Such antibodies are also useful in a method for identifying a viral isolate as a SARS virus comprising reacting said viral isolate or a component thereof with an antibody as provided herein. This can for example be achieved by using purified or non-purified SARS virus or parts thereof (proteins, peptides) using ELISA, RIA, FACS or similar formats of antigen detection assays (Current Protocols in Immunology). Alternatively, infected cells or cell cultures may be used to identify viral antigens using classical immunofluorescence or immunohistochemical techniques. Specifically useful in this respect are antibodies raised against SARS virus proteins which are encoded by a nucleotide sequence comprising one or more of the fragments disclosed in FIG. 2.

Other methods for identifying a viral isolate as a SARS virus comprise reacting said viral isolate or a component thereof with a virus specific nucleic acid according to the invention.

In this way the invention provides a viral isolate identifiable with a method according to the invention as a mammalian virus taxonomically corresponding to a positive-sense single stranded RNA virus identifiable as likely belonging to the SARS virus genus within the family of Coronaviruses.

The method is useful in a method for virologically diagnosing a SARS virus infection of a mammal, said method for example comprising determining in a sample of said mammal the presence of a viral isolate or component thereof by reacting said sample with a nucleic acid or an antibody according to the invention.

Methods of the invention can in principle be performed by using any nucleic acid amplification method, such as the. Polymerase Chain Reaction (PCR; Mullis 1987, U.S. Pat. No. 4,683,195, 4,683,202, en 4,800,159) or by using amplification reactions such as Ligase Chain Reaction (LCR; Barany 1991, Proc. Natl. Acad. Sci. USA 88:189-193; EP Appl. No., 320,308), Self-Sustained Sequence Replication (3SR; Guatelli et al., 1990, Proc. Natl. Acad. Sci. USA 87:1874-1878), Strand Displacement Amplification (SDA; U.S. Pat. Nos. 5,270,184, en 5,455,166), Transcriptional Amplification System (TAS; Kwoh et al., Proc. Natl. Acad. Sci. USA 86:1173-1177), Q-Beta Replicase (Lizardi et al., 1988, Bio/Technology 6:1197), Rolling Circle Amplification (RCA; U.S. Pat. No. 5,871,921), Nucleic Acid Sequence Based Amplification (NASBA), Cleavase Fragment Length Polymorphism (U.S. Pat. No. 5,719,028), Isothermal and Chimeric Primer-initiated Amplification of Nucleic Acid (ICAN), Ramification-extension Amplification Method (RAM; U.S. Pat. Nos. 5,719,028 and 5,942,391) or other suitable methods for amplification of nucleic acids.

In order to amplify a nucleic acid with a small number of mismatches to one or more of the amplification primers, an amplification reaction may be performed under conditions of reduced stringency (e.g. a PCR amplification using an annealing temperature of 38° C., or the presence of 3.5 mM MgCl2). The person skilled in the art will be able to select conditions of suitable stringency.

The primers herein are selected to be “substantially” complementary (i.e. at least 65%, more preferably at least 80% perfectly complementary) to their target regions present on the different strands of each specific sequence to be amplified. It is possible to use primer sequences containing e.g. inositol residues or ambiguous bases or even primers that contain one or more mismatches when compared to the target sequence. In general, sequences that exhibit at least 65%, more preferably at least 80% homology with the target DNA or RNA oligonucleotide sequences, are considered suitable for use in a method of the present invention. Sequence mismatches are also not critical when using low stringency hybridization conditions.

The detection of the amplification products can in principle be accomplished by any suitable method known in the art. The detection fragments may be directly stained or labelled with radioactive labels, antibodies, luminescent dyes, fluorescent dyes, or enzyme reagents. Direct DNA stains include for example intercalating dyes such as acridine orange, ethidium bromide, ethidium monoazide or Hoechst dyes.

Alternatively, the DNA or RNA fragments may be detected by incorporation of labelled dNTP bases into the synthesized fragments. Detection labels which may be associated with nucleotide bases include e.g. fluorescein, cyanine dye or BrdUrd. When using a probe-based detection system, a suitable detection procedure for use in the present invention may for example comprise an enzyme immunoassay (EIA) format (Jacobs et al., 1997, J. Clin. Microbiol. 35, 791-795). For performing a detection by manner of the EIA procedure, either the forward or the reverse primer used in the amplification reaction may comprise a capturing group, such as a biotin group for immobilization of target DNA PCR amplicons on e.g. a streptavidin coated microtiter plate wells for subsequent EIA detection of target DNA-amplicons (see below). The skilled person will understand that other groups for immobilization of target DNA PCR amplicons in an EIA format may be employed.

Probes useful for the detection of the target DNA as disclosed herein preferably bind only to at least a part of the DNA sequence region as amplified by the DNA amplification procedure. Those of skill in the art can prepare suitable probes for detection based on the nucleotide sequence of the target DNA without undue experimentation as set out herein. Also the complementary nucleotide sequences, whether DNA or RNA or chemically synthesized analogs, of the target DNA may suitably be used as type-specific detection probes in a method of the invention, provided that such a complementary strand is amplified in the amplification reaction employed.

Suitable detection procedures for use herein may for example comprise immobilization of the amplicons and probing the DNA sequences thereof by e.g. southern blotting. Other formats may comprise an EIA format as described above. To facilitate the detection of binding, the specific amplicon detection probes may comprise a label moiety such as a fluorophore, a chromophore, an enzyme or a radio-label, so as to facilitate monitoring of binding of the probes to the reaction product of the amplification reaction. Such labels are well-known to those skilled in the art and include, for example, fluorescein isothiocyanate (FITC), β-galactosidase, horseradish peroxidase, streptavidin, biotin, digoxigenin, 35S or 125I. Other examples will be apparent to those skilled in the art.

Detection may also be performed by a so called reverse line blot (RLB) assay, such as for instance described by Van den Brule et al. (2002, J. Clin. Microbiol. 40, 779-787). For this purpose RLB probes are preferably synthesized with a 5′ amino group for subsequent immobilization on e.g. carboxyl-coated nylon membranes. The advantage of an RLB format is the ease of the system and its speed, thus allowing for high throughput sample processing.

The use of nucleic acid probes for the detection of RNA or DNA fragments is well known in the art. Mostly these procedure comprise the hybridization of the target nucleic acid with the probe followed by post-hybridization washings. Specificity is typically the function of post-hybridization washes, the critical factors being the ionic strength and temperature of the final wash solution. For nucleic acid hybrids, the Tm can be approximated from the equation of Meinkoth and Wahl, Anal. Biochem., 138: 267-284 (1984): Tm=81.5° C.+16.6 (log M)+0.41 (% GC)−0.61 (% form)−500/L; where M is the molarity of monovalent cations, % GC is the percentage of guanosine and cytosine nucleotides in the nucleic acid, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, the hybridization and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistm and Molecular Biology—Hybridization with Nucleic Acid Probes, Part I, Chapter 2 “Overview of principles of hybridization and the strategy of nucleic acid probe assays”, Elsevier. New York (1993); and Current Protocols in Molecular Biology, Chapter 2, Ausubel, et al., Eds., Greene Publishing and Wiley-Interscience, New York (1995).

In another aspect, the invention provides oligonucleotide probes for the generic detection of target RNA or DNA. The detection probes herein are selected to be “substantially” complementary to one of the strands of the double stranded nucleic acids generated by an amplification reaction of the invention. Preferably the probes are substantially complementary to the immobilizable, e.g. biotin labelled, antisense strands of the amplicons generated from the target RNA or DNA.

It is allowable for detection probes of the present invention to contain one or more mismatches to their target sequence. In general, sequences that exhibit at least 65%, more preferably at least 80% homology with the target oligonucleotide sequences are considered suitable for use in a method of the present invention. Antibodies, both monoclonal and polyclonal, can also be used for detection purpose in the present invention, for example, in immunoassays in which they can be utilized in liquid phase or bound to a solid phase carrier. In addition, the monoclonal antibodies in these immunoassays can be detectably labeled in various ways. A variety of immunoassay formats may be used to select antibodies specifically reactive with a particular protein (or other analyte). For example, solid-phase ELISA immunoassays are routinely used to select monoclonal antibodies specifically immunoreactive with a protein. See Harlow and Lane, Antibodies, A Laboratory Manual, Cold Spring Harbor Publications, New York (1988), for a description of immunoassay formats and conditions that can be used to determine selective binding. Examples of types of immunoassays that can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be done utilizing immunoassays that are run in either the forward, reverse, or simultaneous modes, including immunohistochemical assays on physiological samples. Those of skill in the art will know, or can readily discern, other immunoassay formats without undue experimentation.

Antibodies can be bound to many different carriers and used to detect the presence of the target molecules. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such using routine experimentation.

The invention also provides a method for serologically diagnosing a SARS virus infection of a mammal comprising determining in a sample of said mammal the presence of an antibody specifically directed against a SARS virus or component thereof by reacting said sample with a proteinaceous molecule or fragment thereof or an antigen according to the invention

Methods and means provided herein are particularly useful in a diagnostic kit for diagnosing a SARS virus infection, be it by virological or serological diagnosis. Such kits or assays may for example comprise a virus, a nucleic acid, a proteinaceous molecule or fragment thereof, an antigen and/or an antibody according to the invention.

Use of a virus, a nucleic acid, a proteinaceous molecule or fragment thereof, an antigen and/or an antibody according to the invention is also provided for the production of a pharmaceutical composition, for example for the treatment or prevention of SARS virus infections and/or for the treatment or prevention of atypical pneumonia, in particular in humans. Preferably a peptide comprising part of the amino acid sequence of the spike protein as depicted in translation 2 with the sequence EMC7 and translation 1 of the RDG seq of FIG. 2, is used for the preparation of a therapeutic or prophylactic peptide. Also preferably, a protein comprising the amino acid sequence of the spike protein as depicted in translation 2 with the sequence EMC7 translation 1 of the RDG seq of FIG. 2, is used for the preparation of a sub-unit vaccine. Furthermore, the nucleocapsid of Cornoviruses, as depicted in the translation of EMC8, in FIG. 2, is known to be particularly useful for eliciting cell-mediated immunity against Coronaviruses and can be used for the preparation of a sub-unit vaccine.

Attenuation of the virus can be achieved by established methods developed for this purpose, including but not limited to the use of related viruses of other species, serial passages through laboratory animals or/and tissue/cell cultures, serial passages through cell cultures at temparutes below 37C (cold-adaption), site directed mutagenesis of molecular clones and exchange of genes or gene fragments between related viruses.

As is shown by Sui et al. (supra) humanised neutralising antibodies have been prepared which have shown to be reactive with the N-terminal 261-672 amino acids of the spike protein of the SARS virus.

A pharmaceutical composition comprising a virus, a nucleic acid, a proteinaceous molecule or fragment thereof, an antigen and/or an antibody according to the invention can for example be used in a method for the treatment or prevention of a SARS virus infection and/or a respiratory illness comprising providing an individual with a pharmaceutical composition according to the invention. This is most useful when said individual comprises a human. Antibodies against SARS virus proteins, especially against the spike protein of SARS virus, preferably against the amino acid sequence as depicted in translation 2 of EMC7 and translation 1 of the RDG seq in FIG. 2, are also useful for prophylactic or therapeutic purposes, as passive vaccines. It is known from other coronaviruses that the spike protein is a very strong antigen and that antibodies against spike protein can be used in prophylactic and therapeutic vaccination.

The invention also provides method to obtain an antiviral agent useful in the treatment of atypical pneumonia comprising establishing a cell culture or experimental animal comprising a virus according to the invention, treating said culture or animal with an candidate antiviral agent, and determining the effect of said agent on said virus or its infection of said culture or animal. An example of such an antiviral agent comprises a SARS virus-neutralising antibody, or functional component thereof, as provided herein, but antiviral agents of other nature are obtained as well. The invention also provides use of an antiviral agent according to the invention for the preparation of a pharmaceutical composition, in particular for the preparation of a pharmaceutical composition for the treatment of atypical pneumonia, especifically when caused by a SARS virus infection, and provides a pharmaceutical composition comprising an antiviral agent according to the invention, useful in a method for the treatment or prevention of a SARS virus infection or atypical pneumonia, said method comprising providing an individual with such a pharmaceutical composition.

Specifically the invention provides a pharmaceutical composition comprising interferon, especially pegylated interferon.

In general all interferon forms would be useful in the present invention, since it is known that all the interferon forms have at least some activity in alleviating (symptoms of) viral infection. However, it is to be understood that preferentially the interferon is used which is derived from the host which is infected with, or which runs the risk of being infected with the virus. Further, most preferred is the use of interferon-alpha, and especially—for coronavirus infections that affect humans, like SARS—human interferon-alpha. Alpha interferon is a natural protein produced by the human body in response to infection. It is also known as interferon alpha-2b. The type I interferon alpha family consists of small proteins that have clinically important anti-infective and anti-tumor activity. It is understood that alpha interferon may be administered alone or in combination with beta interferon or gamma interferon.

Genetic engineering techniques have allowed several companies to mass-produce alpha interferon, which is known as recombinant human alpha interferon, or by abbreviations such as rhIFN or rIFN-alpha. This is marketed under tradenames such as Viraferon (made by Schering-Plough), Roferon-A (by Roche) and Wellferon (by Glaxo SmithKline). Interferon-alpha N3, or Alferon N, is another form of interferon alpha, derived from human leukocytes and containing multiple species of interferon-alpha.

A drawback to the use of interferon-alpha as discussed previously, is the short serum half-life and rapid clearance of the interferon alpha protein. However, it has been shown that the attachment of molecules of polyethylene glycol (PEG) to the interferon, creates a barrier that shields the interferon alfa-2a molecule from being rapidly degraded by proteases in the body and maintains its ability to consistently suppress the target virus over a longer dosage period.

As already discussed above pegylation of proteins such as inteferon is used to prevent rapid removal from the bloodstream and eventually rapid breakdown of the drug. A prolongation of the serum half-life of more than a factor two has been demonstrated (Shannon A. Marshall, Drug Discovery Today Volume 8, Issue 5, March 2003, Pages 212-221). Pegylated IFN alfa-2b has a prolonged serum half-life (40 hours) relative to standard IFN alfa-2b (7-9 hours). The greater size of pegylated IFN alfa-2a acts to reduce glomerular filtration, markedly prolonging its serum half-life (72-96 hours) compared with standard IFN alfa-2a (6-9 hours) (Bruce A. Luxon MD Clinical Therapeutics, Volume 24, Issue 9, September 2002, Pages 1363-1383).

Pegylation of proteins is a standard technique available to a person skilled in the art, and standard pegylated interferons are available commercially from Roche (PEGASYS® (interferon alfa 2a) and Schering-Plough (PEG-Intron A) or in development like PEG-Alfacon, the PEGylated version of Infergen(R) (Interferon alfacon-1) a bio-engineered type I interferon alpha.

Schering-Plough has developed a semi-synthetic form of Intron® A by attaching a 12-kDa mono-methoxy polyethylene glycol to the protein (PEG Intron) which fulfils the requirements of a long-acting interferon alpha protein while providing significant clinical benefits. Pegylation decreases the specific activity of the interferon alpha-2b protein, whilst the potency of PEG Intron, independent of protein concentration; is comparable to the Intron® A standard at both the molecular and cellular level. PEG Intron has enhanced pharmacokinetic profile in both animal and human studies [see Yu-Sen Wang et al., 2002: Advanced Drug Delivery Reviews, Volume 54, Issue 4, 17, Pages 547-570]. In PEGASYS, a 40 kilodalton branched, mobile PEG is covalently bound to the interferon alfa-2a molecule and provides a selectively protective barrier without significantly reducing binding site receptivity.

It is understood that pharmacokinetic behaviour of a pegylated molecule depends on the size of the PEG and the structure of the link between the PEG moiety and the protein (Shannon A. Marshall, Drug Discovery Today Volume 8, Issue 5, March 2003, Pages 212-221). It is known that interferons with smaller PEGs are degraded quickly, requiring more frequent dosing. Thus interferons with larger PEGs are preferred.

Thus the present invention encompasses all types of pegylated interferons or future interferons with yet undisclosed molecule attachments which provide a selectively protective barrier, to shield the interferon from being degraded, without significantly reducing binding site receptivity. Also combinations of different interferons are encompassed in the invention

One of the most preferred embodiments of the present invention is the use of interferon as a prophylactic treatment for the prevention of coronavirus infection. Subjecting apes to a prophylactic or therapeutic treatment either before or during infection with the cornoavirus has a good and useful predictionary value for application of such a prophylaxis or therapy in human subjects.

As is shown in the experimental section administration of interferon before infestation with virus particles greatly delays infection and the effects after infection. It should be understood that the virus challenge given to the test animals is a high dose, which will not or hardly ever occur in ‘natural’ infections. It is understood that viral challenge under ‘natural’ circumstances would equate with a challenge of about 10-105 TCID 50 with a concentration which is much less than that used in the experiment of the invention. Further, the viral challenge in the experiment was for the largest part applied intra-tracheal, i.e. at the place where the virus exerts its main infective activity. Normally, a virus will be encountered in the air that is breathed and this air will firstly pass the nose and/or oral cavity, where it will have a large chance of being filtered out (and stopped) by the epithelium and mucosa of the mouth and/or the nose. Anyhow, the fact that even at the extremely high dose used in our experiments we have been able to show effect of interferon indicates that the effect will even be more pronounced at infective viral doses which are normally encountered. It is therefore believed that prophylactic administration will give a durable and strong protection against infection with coronaviruses.

This is especially important in relation to viruses which are highly infective and/or which have an airborne mode of transmission, such as, for instance, the SARS virus. A prophylactic treatment would be especially welcome for people who run a risk of being infected, such as, in the case of SARS virus, hospital personnel, children, elderly and people having an underlying condition such as diabetes or heart disease, or a weakened immune system.

It remains possible that SARS-CoV infection might be asymptomatic in some, people, or cause nonrespiratory symptoms in others. There is insufficient evidence to exclude the possibility that asymptomatic, or atypical, infected people can transmit the disease. Thus a prophylactic treatment for the prevention of coronavirus infection, like SARS-CoV is indeed essential.

However, our data also show that interferon is also applicable for therapy of coronaviruses, i.e. at the time when virus infection is already established. Our in vivo data show that pathologic effects are at least delayed upon administration of interferon.

Interferon of human and murine origins has been quantified in the art in terms of International Units (“IU”). As used herein, a “unit” of interferon (to be distinguished from “IU”) shall mean the reciprocal of a dilution of interferon-containing material that, as determined by assay, inhibits one-half the number of plaques of a challenge virus, the challenge virus being the vesicular stomatitis virus (“VSV”). So quantified a “unit” of interferon is routinely found to be about one-tenth the quantity of interferon represented by one “IU.” Alternatively, interferon can be quamtitated in μg/kg of body weight.

Interferon is given in doses ranging from 1 μg/kg to 3 μg/kg. When the interferon is pegylated doses can be delivered less frequently. Treatment of a coronavirus disease in accordance with the present invention comprises administering pegylated interferon at a dosage of 0.01-6 μg/kg per day in a dosage form adapted to promote contact of said dosage of interferon with the oral and pharyngeal mucosa of said animal. Preferably, the dosage of interferon is from 0.1-4 μg/kg per day, more preferably 0.3-3 μg/kg per day.

Interferon may be administered by any available means, including but not limited to, oral, intravenous, intramuscular, pulmonary and nasal routes, and wherein said composition is present as a solution, a suspension or an aerosol spray, especially of fine particles.

It is critical that the pegylated interferon be administered in a dosage form adapted to assure maximum contact of the interferon in said dosage form with the oral and pharyngeal mucosa of the human or animal, undergoing treatment. Contact of interferon with the mucosa can be enhanced by maximizing residence time of the treatment solution in the oral or pharyngeal cavity. Thus, best results seem to be achieved in human patients when the patient is requested to hold said solution of interferon in the mouth for a period of time. Contact of interferon with the oral and pharyngeal mucosa and thereafter with the lymphatic system of the treated human or animal avian, rodent is unquestionably the most efficient method administering immunotherapeutic amounts of pegylated interferon.

For example interferon can be administered in either a liquid (solution) or solid dosage form. Thus interferon can be administered dissolved in a buffered aqueous solution typically containing a stabilizing amount (1-5% by weight) of blood serums. Exemplary of a buffered solution suitable as a carrier of interferon administered in accordance with this invention is phosphate buffered saline prepared by standard techniques.

It is also contemplated by the present invention to provide interferon in a solid dosage form such as a lozenge adapted to be dissolved upon contact with saliva in the mouth with or without the assistance of chewing. Such a unitary dosage form is formulated to release about 1 to about 1500 IU of interferon upon dissolution in the mouth for contact with the oral and pharyngeal mucosa. Thus a unitary dosage form of interferon in accordance with this invention can be prepared by art-recognized techniques for forming compressed tablets such as chewable vitamins. Similarly, interferon can be incorporated into starch-based gel formulations to form a lozenge which will dissolve and release interferon for contact with the oral mucosa when held in the mouth. Solid unitary dosage forms of interferon for use in accordance with the present invention can be prepared utilizing art recognized dosage formulation techniques. The pH of such formulations can range from about 4 to about 8.6. Of course, in processing to such unitary dosage forms one should avoid heating a pre-dosage form formulation, after addition of interferon, above about 50° C. Exemplary of a solid dosage form for animal use is a molasses block containing effective amounts of interferon.

Alternatively the interferon can be formulated into flavoured or unflavoured solutions or syrups using a buffered aqueous solution of interferon as a base with added caloric or non-caloric sweeteners, flavour oils and pharmaceutically acceptable surfactant/dispersants.

Also contemplated are methods of gene therapy capable of causing expression of interferon in respiratory or gastric cells for prevention of a coronaviral infection.

Of course, the clinical use of any medicament of the present invention is a clinical decision to be made by the clinician and the exact course of such treatment is left to the clinician's sound discretion, with all such courses of treatment deemed within the bounds of the present invention.

Another preferred embodiment is administration of interferon together with another treatment which is directed to prevent or treat infection with coronaviruses. Such other treatment can for instance be a vaccine, antibody and/or anti-viral agent selected from the group consisting of whole inactivated virus vaccines, attenuated vaccines, sub-unit vaccines, recombinant vaccines, antibody for passive immunization, nucleoside analogs such as ribavirin, RNA-dependent RNA polymerase inhibitors and protease inhibitors.

Use of interferon together with administration of a vaccine will boost the effects of the vaccine. First of all, there is the combination of treatments that will add up to a better effect. However, co-administration of interferon with a vaccine will also enable the immune response to vaccination to have more effect. Normally the immune response is slow and it takes a few days to come to a high enough titer of antibodies to be able to effectively combat virus particles. When no interferon is co-administered the virus would have had the chance to multiply to enormous amounts, which cannot be overcome by the immune response. With interferon, however, the amounts of the virus will remain absent or low and any infective virus outburst (if any at all) can easily be handled by the immune system.

Treatment to prevent and/or treat infection with coronaviruses can also comprise combination treatments with other antiviral compounds, such as, for instance, nucleoside-based compounds such as ribavirin (e.g. Rebetol® (ribavirin, USP). These compounds act through interfering with the viral replication by presenting nucleosides which are built in during viral replication, but which either prevent formation of viral proteins or which do not yield functional proteins. Co-administration of interferon will even more slow down viral replication. Combinations of ribavirin and forms of interferon can help to reduce viral load.

Another disease condition responding to treatment in accordance with the present invention is neoplastic disease. Thus, the administration of interferon in accordance with the above description can, alone or in combination with other drugs or therapy, help effect remission of cancers such as malignant lymphoma, melanoma, mesothelioma, Burkitt lymphoma and nasopharyngeal carcinoma and other neoplastic diseases, especially those of known or suspected viral etiology and diseases such as Hodgkin's Disease and leukemia.

Other disease conditions responding to treatment in accordance with the present invention are infectious diseases of coronaviral origin in human, avian, porcine, canine and feline species. Human coronavirises are coronivirus 229E and the newly discovered coronavirus HcoV-NL (see European patent application 03078772.5. Several other coronaviruses can cause fatal systemic diseases in animals, including feline infectious peritonitis virus (FIPV), hemagglutinating encephalomyelitis virus (HEV) of swine, and some strains of avian infectious bronchitis virus (IBV) and mouse hepatitis virus (MHV). These coronaviruses can replicate in liver, lung, kidney, gut, spleen, brain, spinal cord, retina, and other tissues. Immunopathology plays a role in tissue damage in MHV and FIPV, and cytokines are responsible for some signs of disease. Significantly, in cats with persistent, inapparent infection with feline enterotropic coronavirus, virulent virus mutants can arise and cause fatal infectious peritonitis, a systemic disease.

The invention also comprises an animal model usable for testing of prophylactic and/or therapeutic methods and/or preparations. It has appeared that apes can be infected with the SARS virus, thereby showing clinical symptoms, and more importantly, similar tissue morphology as found in humans suffering from atypical pneumonia caused by the SARS virus. Subjecting apes to a prophylactic or therapeutic treatment either before or during infection with the virus will have a good and useful predictionary value for application of such a prophylaxis or therapy in human subjects.

The invention is further explained in the Examples without limiting it thereto.

### FIGURE LEGENDS

FIG. 1: Phylogenetic relationship for the nucleotide sequences of isolate HK39849 with its closest relatives genetically. Phylogenetic trees were generated by maximum likelihood analyses using 100 bootstraps and 3 jumbles. The scale representing the number of nucleotide changes is shown for each tree.

FIG. 2: Nucleotide sequences from 13 clones of parts of the SARS virus. Also included are the putative polypeptide sequences of polypeptides and alignments of the putative polypeptides with that of another member of the Coronoviridae family, where possible.

FIG. 3: Schematic map of the SARS virus genome, indicating the position of the nucleotide sequences of FIG. 2 relative to the genome and a putative indication of the open reading frames of the genome based on analogy with other coronaviruses. The gene structure for the region between the Spike and Nucleocapsid is uncertain. EMC1-EMC14 and RDG 1: sequences as provided in FIG. 2. CDC and BIN1-2: sequences were provided through personal communication from the CDC (Dr. W. Bellini, Centers for Disease Control & Prevention, National Centers for Infectious Diseases, 1600 Clifton Road, Atlanta, Ga. 30333, USA) and BNI (Dr. C . Drosten and Prof. Dr. H. Schmitz, Bernard Nocht Institute, Bernard-Nocht Str. 74, D-20359 Hamburg, Germany), respectively.

FIG. 4: Amino acid comparison of the N-terminus of the S-protein of the SARS virus and closely related coronaviruses. HCV OC43=human coronavirus isolate OC43; MHV A59=murine hepatitis virus isolate A59, BCV=bovine corona virus.

FIG. 5: Negative contrast EM photograph of SARS virus obtained from concentrated supernatant of infected cell cultures.

FIG. 6: Infection with SARS-coronavirus causes pulmonary and renal lesions in cynomolgus macaques. Formalin-fixed, paraffin-embedded tissues were stained with haematoxylin and eosin and examined by light microscopy. There is diffuse alveolar damage of the lung (a), and the alveolar luimina (b) are flooded with highly proteinaceous exudate admixed with inflammatory cells and cellular debris. In the lumen of a bronchiole (c) and in the surrounding lung parenchyma are several multinucleated syncytial cells (arrowheads). The renal collecting tubules (d) contain similar multinucleated syncytial cells. Original magnifications: a×12.5; b×50; c×100; d×250.

FIG. 7: Infection of domestic cats and ferrets with SCV. Cats (A, n=6) and ferrets (B, n=6) were infected with 10⁶ TCID₅₀ via the respiratory route and secretion of SCV in pharyngeal swabs was quantified by real time PCR. Four animals per group were euthanised at day 4 while the other two were analysed till day 28. SCV secretion in non-infected cats (C, n=2) and non-infected ferrets (D, n=2) exposed to SCV infected cats and ferrets, respectively. Real time PCR results are shown relative to a titrated SCV standard and shown as TCID₅₀/ml (N.D. not done).

FIG. 8: Detection of SCV in postmortem tissues of experimentally SCV infected cats and ferrets

FIG. 9: Effect of pegylated IFN-α on SARS Coronavirus (SCV) replication in macaques. SCV detection in pharyngeal swabs (days 0, 2 and 4 after infection, closed bars) and lungs (day 4, open bars) taken from cynomolgus monkeys treated with PBS (A), PEG-Intron at days −3, −1, +1 and +3 (B and C) and PEG-Intron at days +1 and +3 (D) after SCV infection. Individual macaques are shown (n=2 per group). Virus isolation (VI) results are indicated in the lower part of the panel whereas real time PCR results are shown in the upper part of the panels (n.a., not available).

FIG. 10 Nucleotide sequence of SARS Corona virus Genbank accession nr. AY274119

FIG. 11 Antiviral activity of pegylated IFN-α against SCV in vitro and its biological activity in cynomolgus macaques. (a) Effect of pegylated IFN-α against SCV infection in vitro. Similar results were obtained in 3 separate experiments. (b) Pharmacokinetic analysis of pegylated IFN-α in macaques treated with PBS (control group; open squares, n=4) or pegylated IFN-α (prophylactic group; closed squares, n=4) at days −3 and −1. (c) Induction of neopterin in macaques treated with PBS (control group; open squares, n=4) or pegylated IFN-α (prophylactic group; closed squares, n=4) at days −3 and −1. Data are expressed as mean±s.d.; **, P<0.01 versus control.

FIG. 12 Effect of pegylated IFN-α on SCV excretion in cynomolgus macaques. SCV detection in pharyngeal swabs taken at 0, 2, or 4 d.p.i. from macaques treated with PBS (control group, n=4), pegylated IFN-α prophylactically (n=6) or post-exposure (n=4). Data are expressed as mean±s.d.; *, P<0.05 versus control group at 2 d.p.i., **, P<0.01 versus control group at 2 d.p.i.

FIG. 13 Effect of pegylated IFN-α on SCV replication, viral antigen expression and histological lesions in the lungs of SCV-infected cynomolgus macaques. (a) SCV titration of lung homogenates. (b) Immunohistochemical detection of SCV-infected cells in lung sections. (c) Histopathological score of lung sections. SCV-infected macaques were treated with pegylated IFN-α prophylactically (n=4) or post-exposure (n=4), or treated with PBS (control group, n=4). Data are expressed as mean±s.d.; *, P<0.05 versus control group; **, P<0.01 versus control group.

### EXAMPLES

### Example 1

### Virus Isolation and Characterisation

Isolation

Isolate HK39849 was isolated from a hospitalised SARS patient by throat swab and inoculated into a culture of Vero-E6 cells. A sample of the supernatant from these infected cells provided by Dr. M. Peiris (Queeen Mary Hospital Faculty of Medicine, Hong Kong University, Honk Kong) was used to inoculate VERO-118 cells and cell culture supernatant from these cells was aliquoted and frozen after one passage.

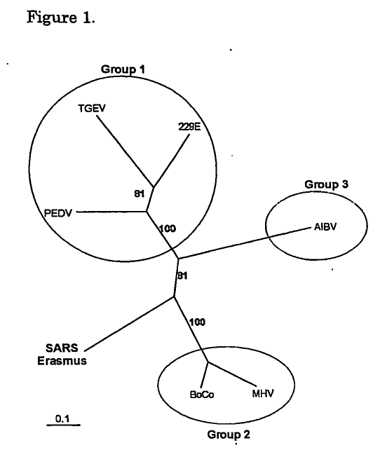

We isolated RNA from the virus-containing cell culture supernatant and subjected it to RNA arbitrarily primed PCR (RAP-PCR) essentially as described by Welsh & McClelland (NAR 18:7213; PNAS USA 90:10710, 1993). Virus in the culture supernatants was purified on continuous 20-60% sucrose gradients. The gradient fractions were inspected for virus-like particles by EM, and RNA was isolated from the fraction containing, in which the most nucleocapsids were observed. Equivalent amounts of RNA isolated from virus fractions were used for RAP-PCR, after which samples were run side by side on a 3% NuSieve agarose gel. Differentially displayed bands ranging in size from 200-1500 base pairs specific for the unidentified virus were subsequently purified from the gel, cloned in plasmid pCR2.1 (Invitrogen) and sequenced with vector-specific primers. When we used these sequences to search for homologies against sequences in the Genbank database using the BLAST software (www.ncbi.nlm.nih.gov/BLAST/) which yielded resemblance to virus sequences of the coronaviruses displayed in the phylogenetic tree of FIG. 1.

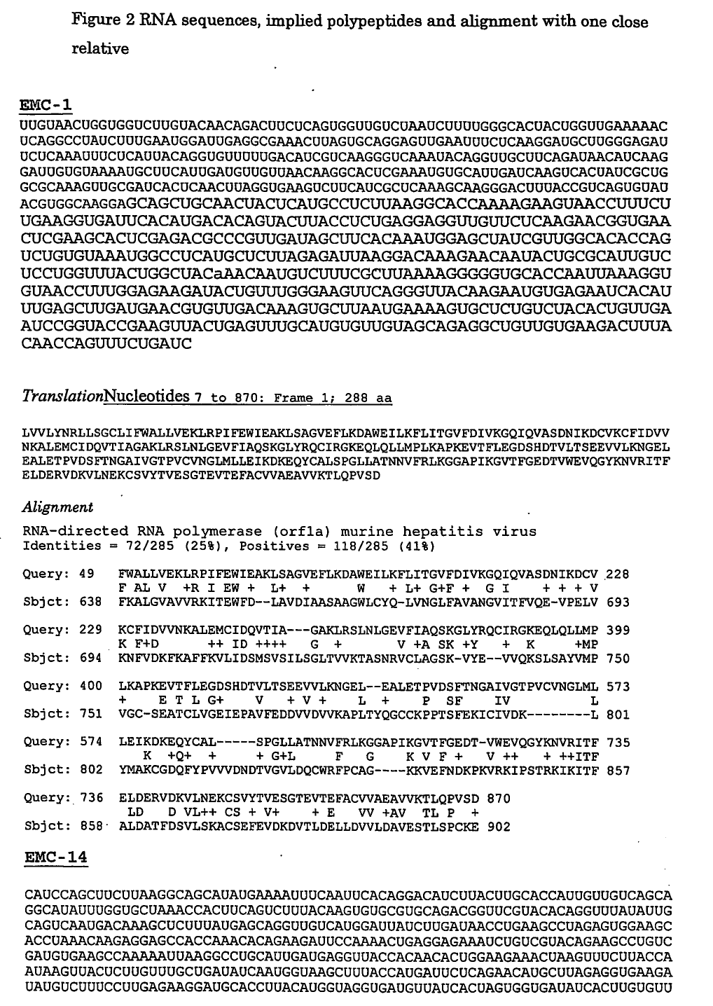

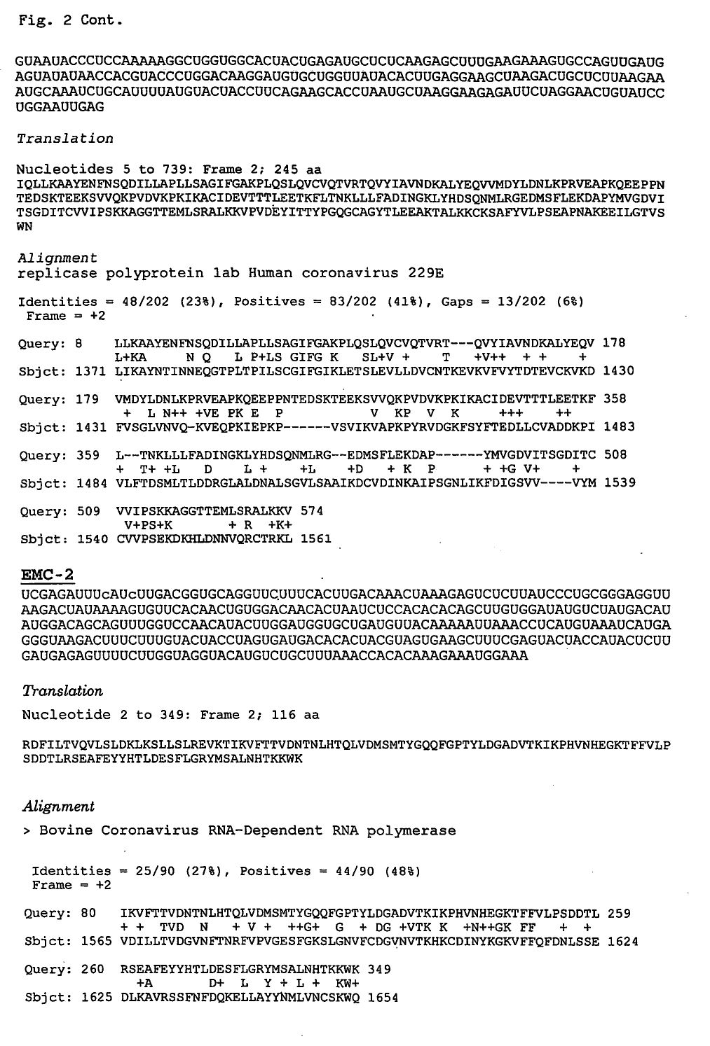

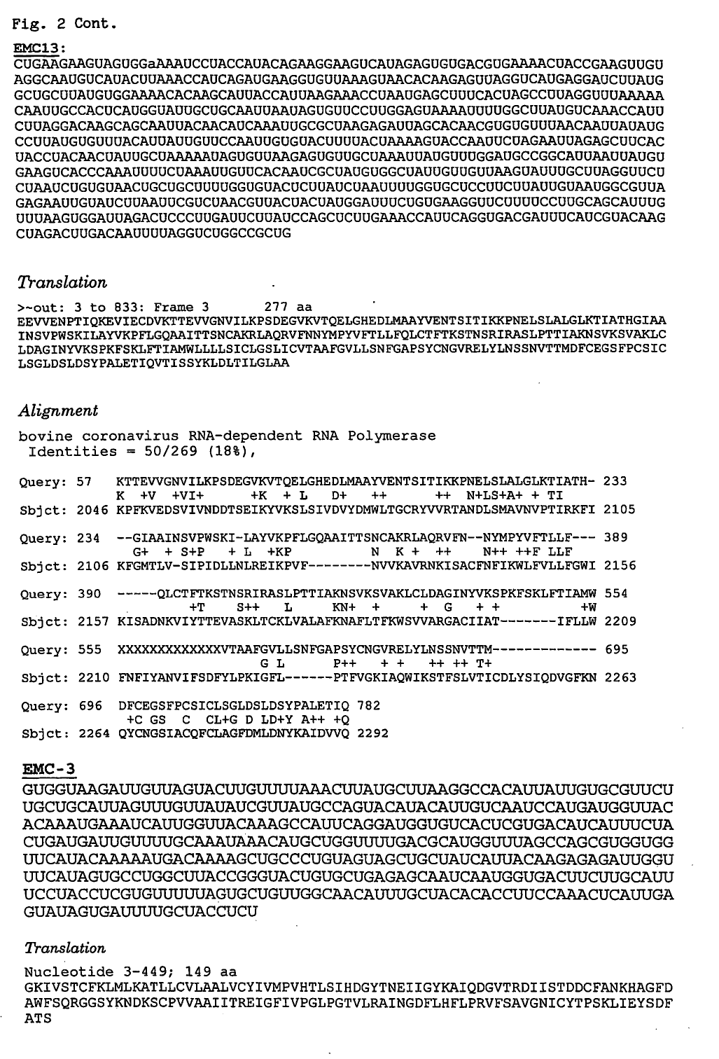

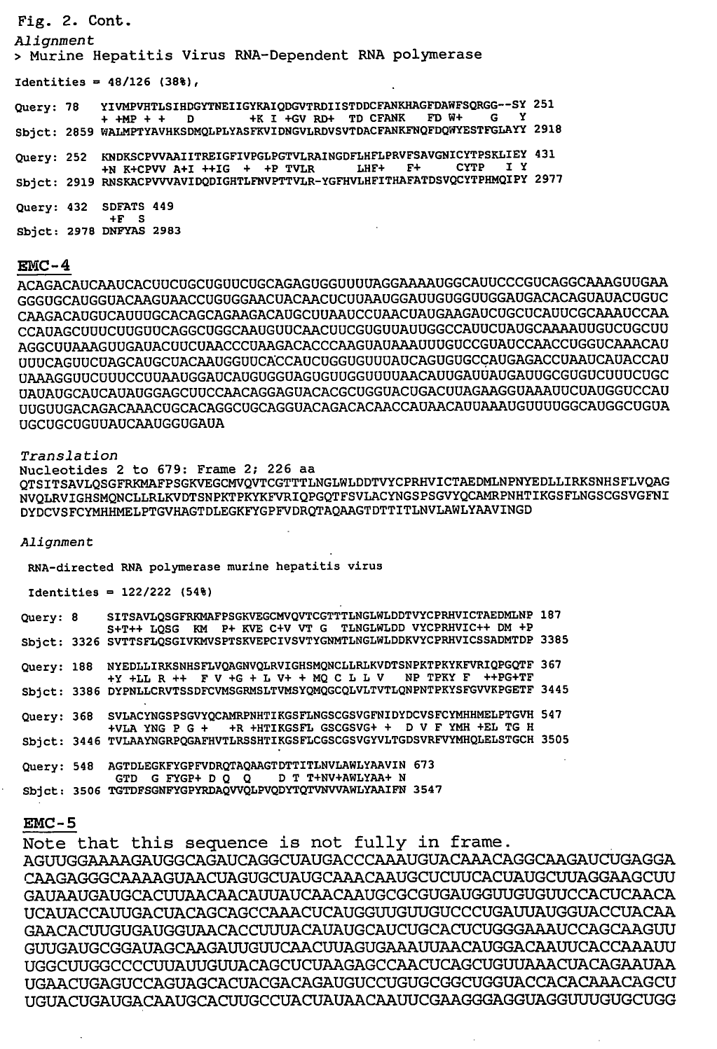

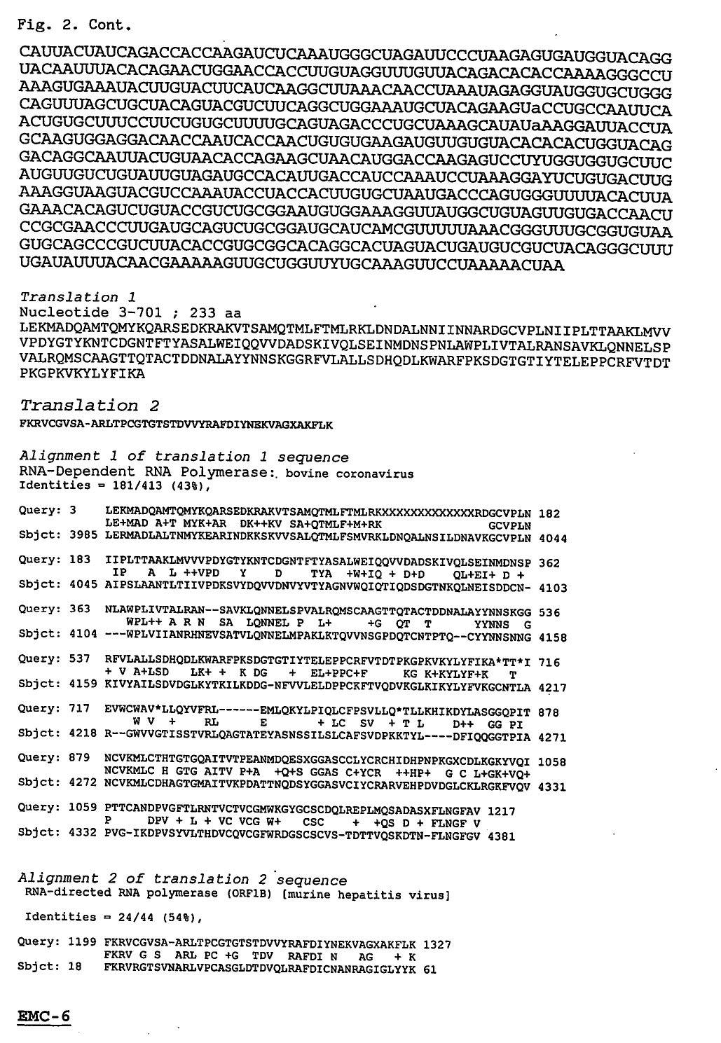

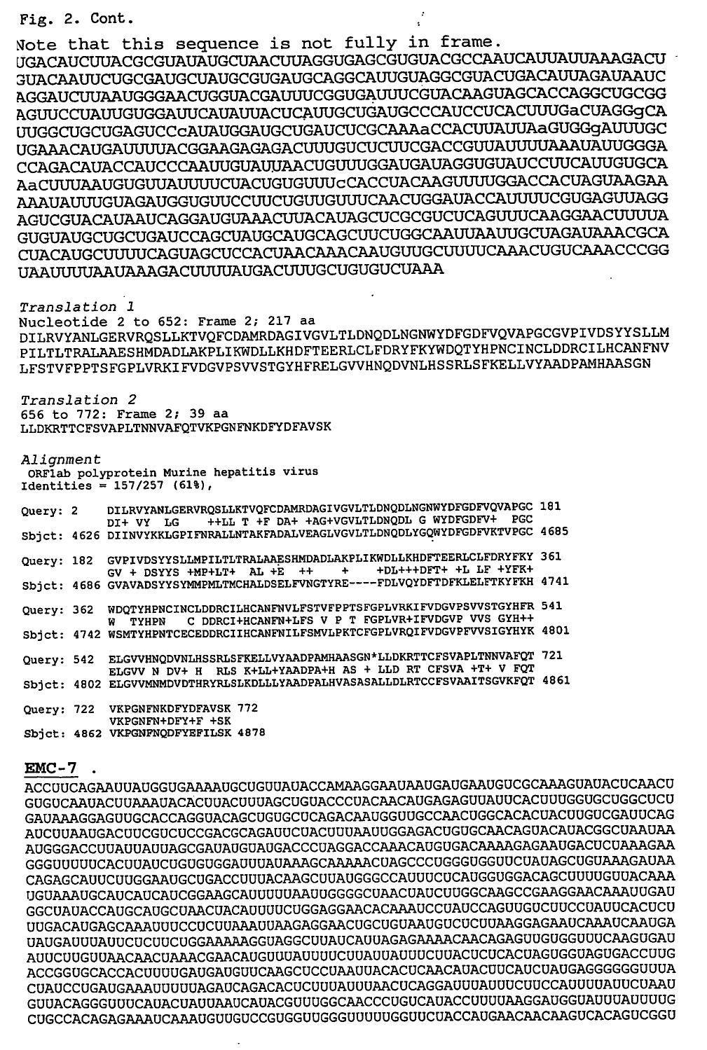

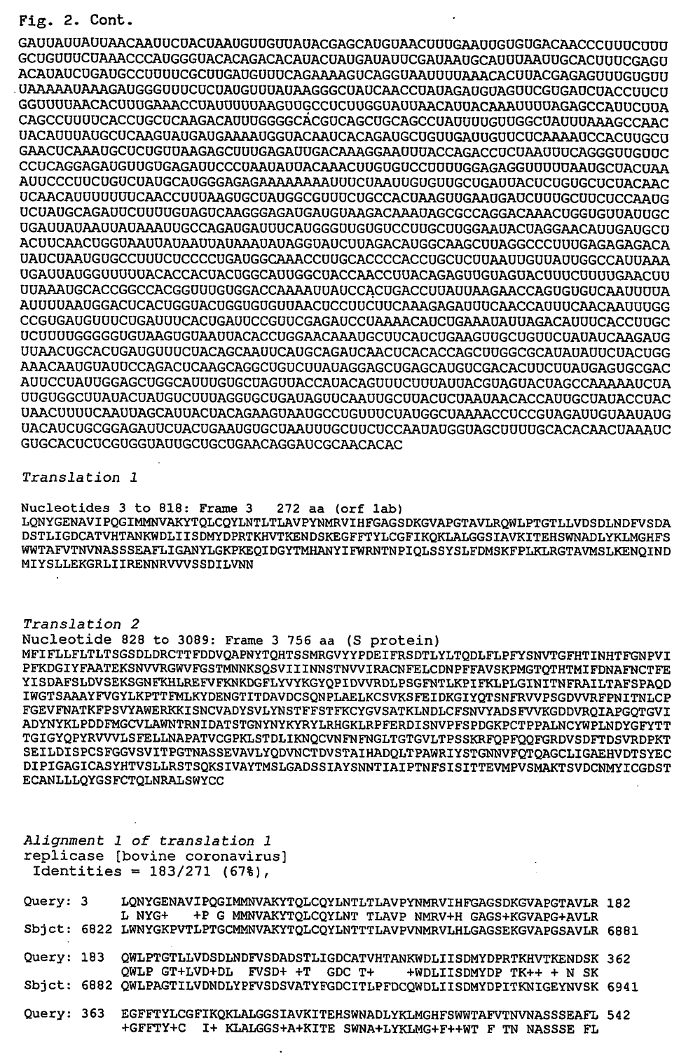

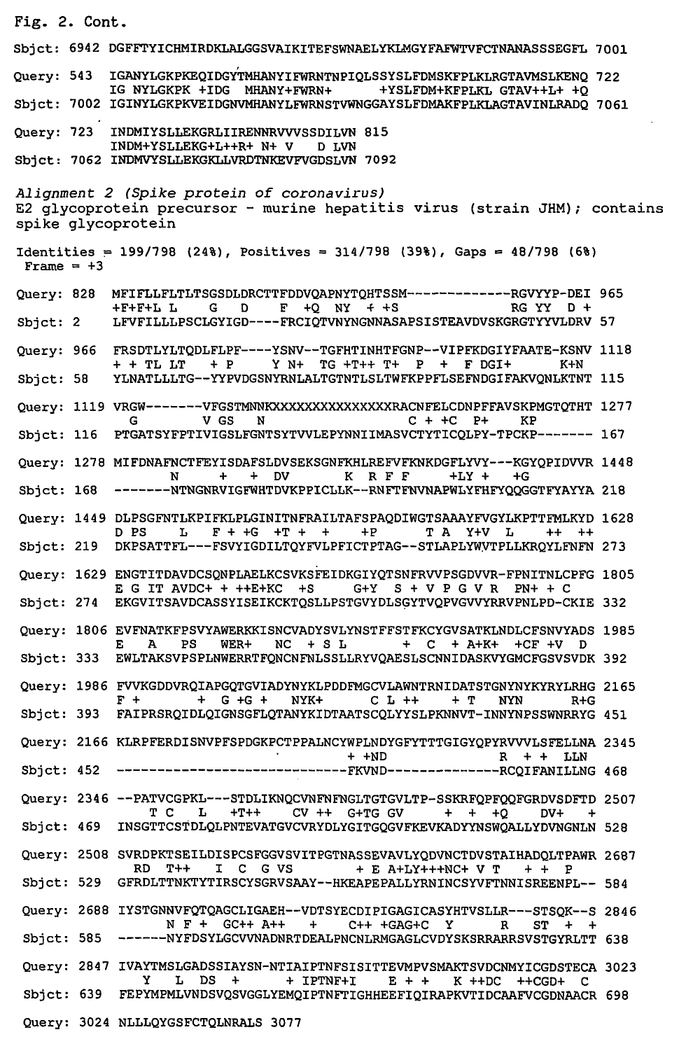

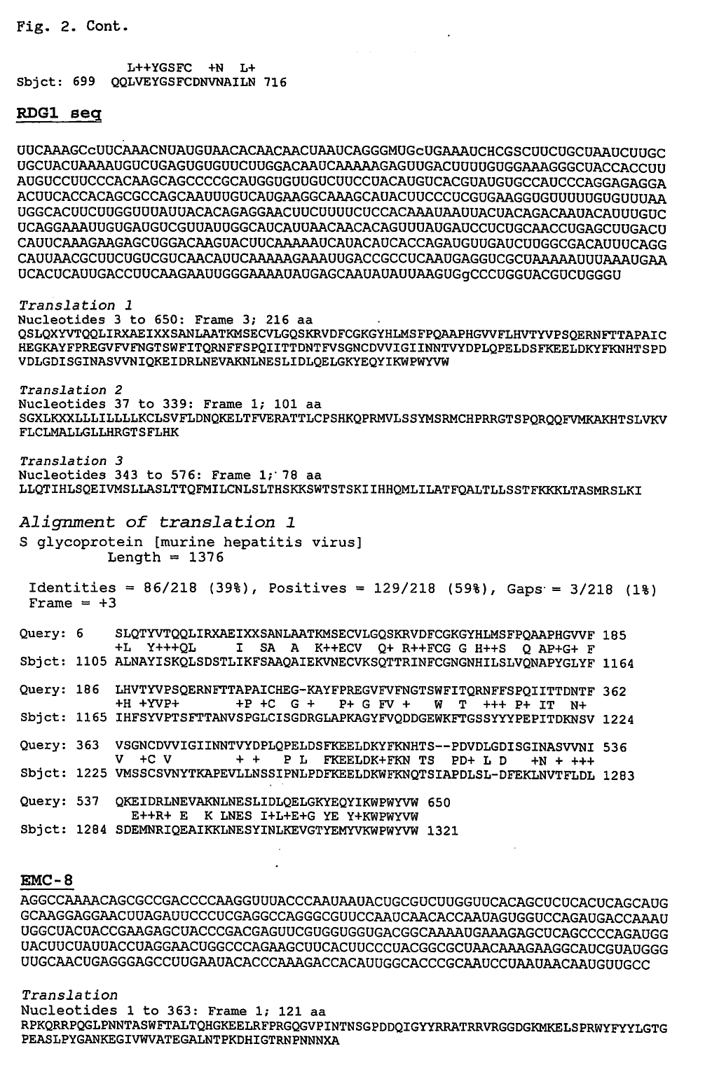

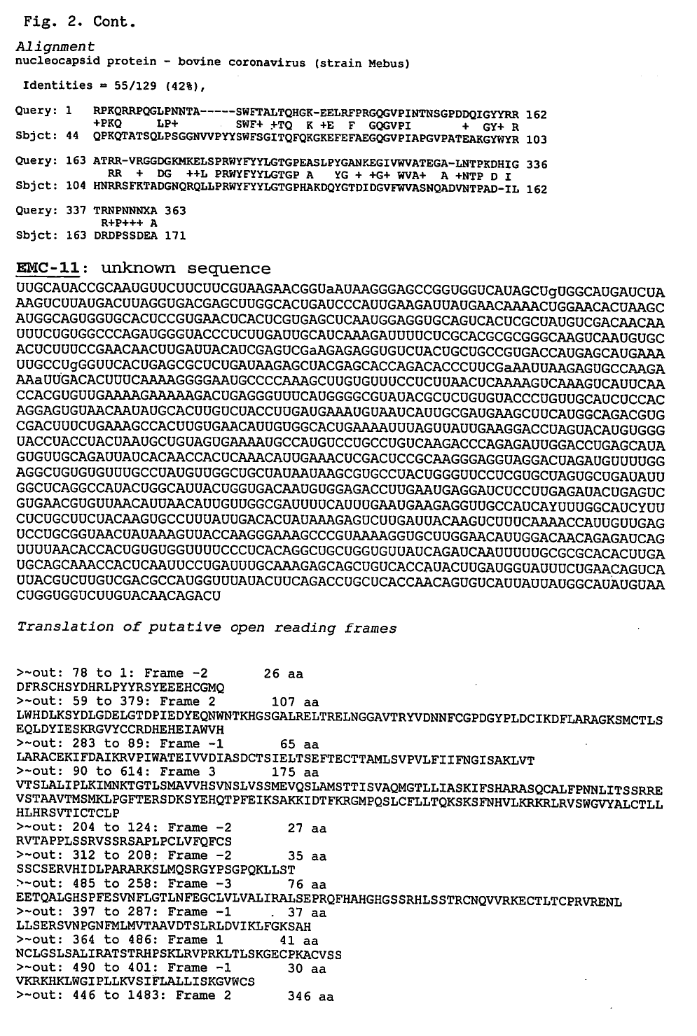

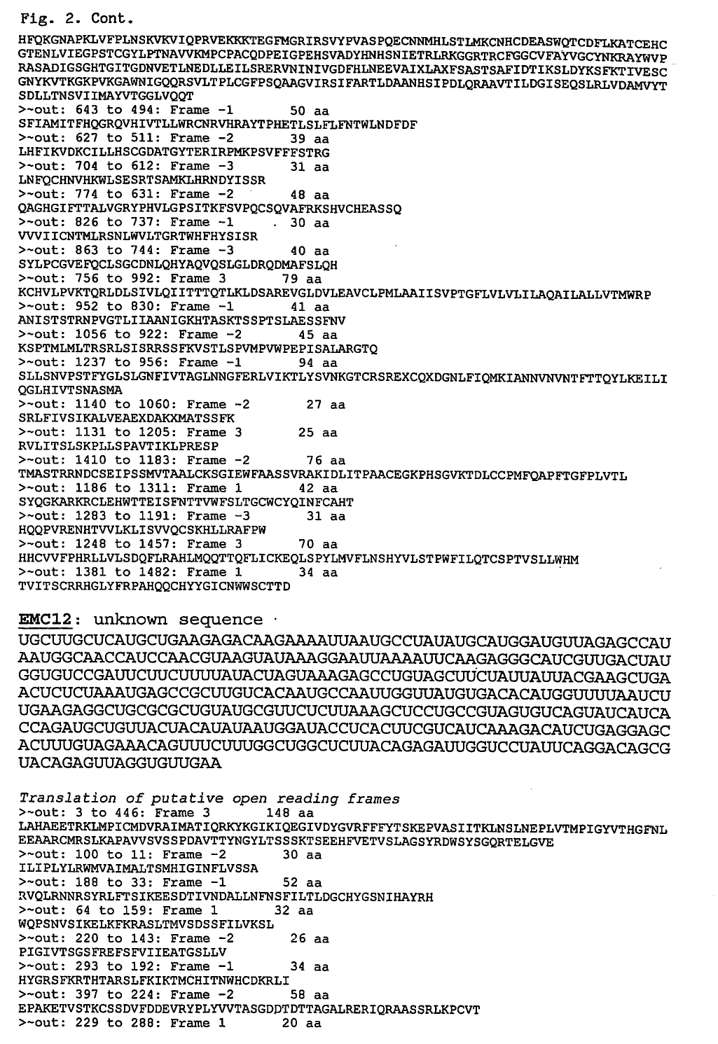

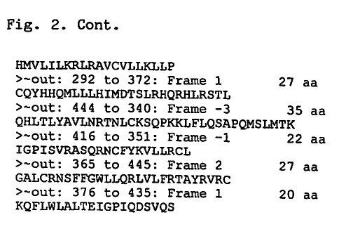

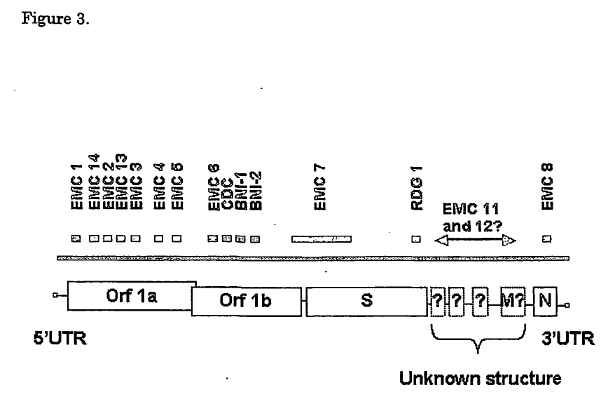

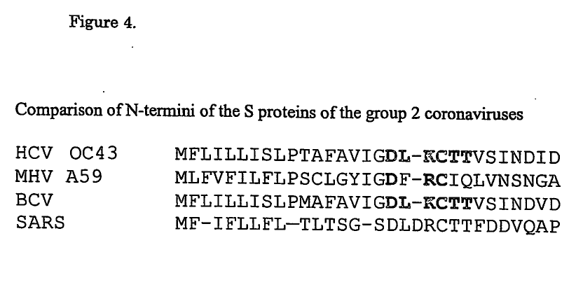

Eight of these fragments (EMC 1-6, 13 and 14) were located in the ORF coding for the viral polymerase (ORF lab), one (EMC-7) spanned the 3′ end of ORFlab and reached into the 5′ end of spike protein region; EMC-10 overlapped the 3′ end of EMC-7 and therefore also codes part of the S protein region and EMC 9 encodes a region downstream of EMC-10; by use of primers to sequences within EMC10 and EMC9 (see below), the region between these two sequences was amplified by PCR and sequenced. The full contiguous region has been incoporated into EMC7 in FIG. 2; a further sequence (RDG1 in FIG. 2) encodes the 3′ end of the Spike protein. A further sequence (EMC8) spanned part of the Nucleocapsid coding sequence. The remaining three sequences (EMC9, 11 and 12) have in the meantime been found to be regions of the orf 1ab/replicase, where emc 9 is incorporated in emc 11. This has not yet been reflected in FIG. 3.

Phylogeny

BLAST searches using nucleotide sequences obtained from the unidentified virus isolate revealed homologies primarily with members of the Coronaviridae. As an indication for the relation between the newly identified virus isolate and other coronaviruses a phylogenetic tree was constructed based on the sequence information obtained (FIG. 1).

Materials and Methods

Specimen Collection

Virus was collected from SARS patients using throat swabs and from experimentally infected monkeys (throat and nasal swabs, serum, plasma and faeces)

Virus Isolation and Culture

Throat swabs were dipped into a culture of Vero-E6 cells and incubated for 1-4 days. Cell culture supernatant was clarified by centrifugation and filtered through a 0.45 micrometre filter, before beings stored frozen. The virus was subsequently propagated in Vero-118 cells.

Antigen Detection by Indirect IFA

Samples from experimentally infected monkeys was cultured on Vero-118 cells in 24 well plates containing glass slides. These glass slides were washed with PBS and fixed in aceton for 1 minute at room temperature. After washing with PBS the slides were incubated for 30 minutes at 37° C. with SARS-antibody containing serum from SARS patients. After washing off the human serum in PBS, the slides were incubated at 37° C. for 30 minutes with FITC labeled anti-human antibodies. After three washes in PBS and one in tap water, the slides were included in a glycerol/PBS solution (Citifluor, UKC, Canterbury, UK) and covered. The slides were analysed using an Axioscop fluorescence microscope (Carl Zeiss B.V., Weesp, the Netherlands).

Detection of Antibodies in Humans by Indirect IFA

Virus was cultured on Vero-118 cells in 24 well plates containing glass slides. These glass slides were washed with PBS and fixed in aceton for 1 minute at room temperature. After washing with PBS the slides were incubated for 30 minutes at 37° C. with SARS-antibody containing serum from SARS patients. After washing off the human serum in PBS, the slides were incubated at 37° C. for 30 minutes with FITC labeled anti-human antibodies. After three washes in PBS and one in tap water, the slides were included in a glycerol/PBS solution (Citifluor, UKC, Canterbury, UK) and covered. The slides were analysed using an Axioscop fluorescence microscope (Carl Zeiss B.V., Weesp, the Netherlands

Detection of Antibodies in Humans by ELISA

Patient Samples.

4 samples of patients with SARS disease, 8 samples of patients from routine serological virology; samples from an experimentally infected monkey (preserum, 9 and 12 days after infection).

The Conjugate.

Whole virus was used as the conjugate. Tissue culture supernatant from infected Vero cells were pelleted through 20% sucrose onto a 60% sucrose cushion. The virus was then pelleted through 20% sucrose and resuspended in PBS/1% NP40. After dialysis using PBS, the virus was The conjugated to horseradish peroxidase by standard techniques was tested in 3 concentrations (diluted in dilution buffer 9000-03, 1:100, 1:400 and 1:1600), both on polyvalent anti-IgM code MCB0201 (cross-reactive with monkey) and monoclonal anti-IgM, code 9000-62 (non-crossreactive with monkey).

Sera were diluted 1:200 in serum diluent (code 9000-03), monkey 775 was diluted 1:100, 1:200 and 1:400.

Serum incubation one hour at 37° C., conjugate incubation one hour at 37° C., and TMB (ready to use): 30 minutes at room temperature. The reaction was stopped with sulphuric acid (0.5M).

Virus Characterisation

For EM analyses, virus was concentrated from infected cell culture supernatants in a micro-centrifuge at 4° C. at 17000×g, after which the pellet was resuspended in PBS and inspected by negative contrast EM

RNA Isolation

RNA was isolated from the supernatant of infected cell cultures or sucrose gradient fractions using a High Pure RNA Isolation kit according to instructions from the manufacturer (Roche Diagnostics, Almere, The Netherlands).

RT-PCR

A one-step RT-PCR was performed in 50 μl reactions containing 50 mM Tris.HCl pH 8.5, 50 mM NaCl, 4 mM MgCl₂, 2 mM dithiotreitol, 200 μM each DNTP, 10 units recombinant RNAsin (Promega, Leiden, the Netherlands), 10 units AMV RT (Promega, Leiden, The Netherlands), 5 units Amplitaq Gold DNA polymerase (PE Biosystems, Nieuwerkerk aan de Ijssel, The Netherlands) and 5 μl RNA. Cycling conditions were 45 min. at 42° C. and 7 min. at 95° C. once, 1 min at 95° C., 2 min. at 42° C. and 3 min. at 72° C. repeated 40 times and 10 min. at 72° C. once.

Primers used for diagnostic PCR:

SARS fwd2: ggtggaacatcatccggtgat
SARS rev2: agcctgtgttgtagattgcgg

These primers amplify a 149 bp fragment of the polymerase gene (orf 1ab)

RF 999: TTTAAACACTTACGAGAGTTTGTG
RF997: GGACACAACCCATGAAATCATCTGG

These primers amplify a region of 728 bp in the spike glycoprotein gene (S)

RF998: AGACATATCTAATGTGCCTTTCTCC
RF1002: AAGCTCGTCACCTAAGTCATAAGAC
(from EMC11 sequence)

The combination of RF998/RF1002 primers enabled us to sequence the 3′ end of EMC7-RF998 is a specific primer withing EMC7 whereas EMC1002 acted as a random primer.

RT-PCR, gel purification and direct sequencing were performed as described above.

RAP-PCR

RAP-PCR was performed essentially as described by Welsh & McClelland (Nuc. Acid Res. 18:7213, 1990; Proc. Natl. Acad. Sci. USA 90:10710 1993). The oligonucleotide sequences are described in addenda 2. For the RT reaction, 2 μl RNA was used in a 10 μl reaction containing 10 ng/μl oligonucleotide, 10 mM dithiotreitol, 500 μm each dNTP, 25 mM Tris-HCl pH 8.3, 75 mM KCl and 3 mM MgCl2. The reaction mixture was incubated for 5 min. at 70° C. and 5 min. at 37° C., after which 200 units Superscript RT enzyme (LifeTechnologies) were added. The incubation at 37° C. was continued for 55 min. and the reaction terminated by a 5 min. incubation at 72° C. The RT mixture was diluted to give a 50 μl PCR reaction containing 8 ng/μl oligonucleotide, 300 μm each DNTP, 15 mM Tris-HCL pH 8.3, 65 mM KCl, 3.0 mM MgCL₂ and 5 units Taq DNA polymerase (PE Biosystems). Cycling conditions were 5 min. at 94° C., 5 min. at 40° C. and 1 min. at 72° C. once, followed by 1 min. at 94° C., 2 min. at 56° C. and 1 min. at 72° C. repeated 40 times and 5 min. at 72° C. once. After RAP-PCR, 15 μl the RT-PCR products were run side by side on a 3% NuSieve agarose gel (FMC BioProducts, Heerhugowaard, The Netherlands). Differentially displayed fragments were purified from the gel with Qiaquick Gel Extraction kit (Qiagen, Leusden, The Netherlands) and cloned in pCR2.1 vector (Invitrogen, Groningen, The Netherlands) according to instructions from the manufacterer.

Sequence Analysis

RAP-PCR products cloned in vector pCR2.1 (Invitrogen) were sequenced with M13-specific oligonucleotides. DNA fragments obtained by RT-PCR were purified from agarose gels using Qiaquick Gel Extraction kit (Qiagen, Leusden, The Netherlands), and sequenced directly with the same oligonucleotides used for PCR. Sequence analyses were performed using a Dyenamic ET terminator sequencing kit (Amersham Pharmacia Biotech, Roosendaal, The Netherlands) and an ABI 373 automatic DNA sequencer (PE Biosystem). All techniques were performed according to the instructions of the manufacturer.

RT-PCR for Diagnosing SARS Virus.

For the amplification of the SARS virus' genetic material, we used primers:

SARS fwd2: ggtggaacatcatccggtgat
SARS rev2: agcctgtgttgtagattgcgg

These primers amplify a 149 bp fragment of the polymerase gene (orf 1ab)

RF 999: TTTAAACACTTACGAGAGTTTGTG
RF997: GGACACAACCCATGAAATCATCTGG

These primers amplify a region of 728 bp in the spike glycoprotein gene (S)

These primers amplify a 149 bp fragment of the polymerase gene (orf 1ab) RT-PCR, gel purification and direct sequencing were performed as described above.

Phylogenetic Analyses

For all phylogenetic trees, DNA sequences were alligned using the ClustalW software package and maximum likelihood trees were generated using the DNA-ML software package of the Phylip 3.5 program using 100 bootstraps and 3 jumbles¹⁵. Previously published sequences for TGEV, PEDV, 229E, AIBV, BoCo and MHV that were used for the generation of phylogenetic trees are available from Genbank

### Example 2

### Methods to Identify SARS Virus

Specimen Collection

In order to find virus isolates nasopharyngeal aspirates, throat and nasal swabs, broncheo alveolar lavages, serum and plasma samples, and stools preferably from mammals such as humans, carnivores (dogs, cats, mustellits, seals etc.), horses, ruminants (cattle, sheep, goats etc.), pigs, rabbits, birds (poultry, ostriches, etc) should be examined. From birds cloaca swabs and droppings can be examined as well. Sera should be collected for immunological assays, such as ELISA, molecular-based assays, such as RT-PCR and virus neutralisation assays.

Collected virus specimens were diluted with 5 ml Dulbecco MEM medium (BioWhittaker, Walkersville, Md.) and thoroughly mixed on a vortex mixer for one minute. The suspension was thus centrifuged for ten minutes at 840×g. The sediment was spread on a multispot slide (Nutacon, Leimuiden, The Netherlands) for immunofluorescence techniques, and the supernatant was used for virus isolation.

Virus Isolation

For virus isolation Vero-118 cells or tMK cells (RIVM, Bilthoven, The Netherlands) were cultured in 24 well plates containing glass slides (Costar, Cambridge, UK), with the medium described below supplemented with 10% fetal bovine serum (BioWhittaker, Vervier, Belgium). Before inoculation the plates were washed with PBS and supplied with Eagle's MEM with Hanks' salt (ICN, Costa Mesa, Calif.) supplemented with 0.52/liter gram NaHCOs, 0.025 M Hepes (Biowhittaker), 2 mM L-glutamine (Biowhittaker), 200 units/liter penicilline, 200 μg/liter streptomycine (Biowhittaker), 1 gram/liter lactalbumine (Sigma-Aldrich, Zwijndrecht, The Netherlands), 2.0 gram/liter D-glucose Merck, Amsterdam, The Netherlands), 10 gram/liter peptone (Oxoid, Haarlem, The Netherlands) and 0.02% trypsine (Life Technologies, Bethesda, Md.). The plates were inoculated with supernatant of the patient samples, 0.2 ml per well in triplicate, followed by centrifuging at 840×g for one hour. After inoculation the plates were incubated at 37° C. for a maximum of 1-3 days and cultures were checked daily for CPE. Extensive CPE was generally observed within 24 hours and included detachment of cells from the monolayer.

Virus Culture of SARS

Sub-confluent monolayers of tMK cells or Vero clone 118 cells in media as described above were inoculated with supernatants of samples that displayed CPE or with samples taken from patient or artificially infected monkeys.

Virus Characterisation

For EM analyses, virus was concentrated from infected cell culture supernatants in a micro-centrifuge at 4° C. at 17000×g, after which the pellet was resuspended in PBS and inspected by negative contrast EM.

Antigen Detection by Indirect IFA

Virus was cultured on Vero-118 cells in 24 well slides containing glass slides. These glass slides were washed with PBS and fixed in aceton for 1 minute at room temperature.

After washing with PBS the slides were incubated for 30 minutes at 37° C. with SARS patient serum. We used patient serum, but antibodies can be raised in various animals, such as ferrets, goats and rabbits (for polyclonal antibodies) and mice and hamsters (for monoclonal antibodies), and the working dilution of the antibody can vary for each immunisation. After three washes with PBS and one wash with tap water, the slides were incubated at 37° C. for 30 minutes with FTIC. labeled goat-anti-human antibodies. After three washes in PBS and one in tap water, the slides were included in a glycerol/PBS solution (Citifluor, UKC, Canterbury, UK) and covered. The slides were analysed using an Axioscop fluorescence microscope (Carl Zeiss B.V., Weesp, the Netherlands).

Detection of Antibodies in Humans by Indirect IFA

For the detection of virus specific antibodies, SARS virus-infected Vero cells were fixed with acetone on coverslips (as described above), washed with PBS and incubated 30 minutes at 37° C. with serum samples at a 1 to 16 dilution. After two washes with PBS and one with tap water, the slides were incubated 30 minutes at 37° C. with FITC-labelled secondary antibodies to human antibodies (Dako). Slides were processed as described above.

Antibodies can be labelled directly with a fluorescent dye, which will result in a direct immuno fluorescence assay. FITC can be replaced with any fluorescent dye. This technique can be applied to antibodies in other animals such as mammals, ruminants, birds or other species, assuming the secondary antibody to the appropriate species is used.

Detection of Antibodies in Humans by ELISA

Patient Samples.

4 samples of patients with SARS; 8 samples of patients from routine serological virology; samples from an experimentally infected monkey (preserum and 9 days after infection).

The Conjugate.

The conjugate was tested at a number of concentrations, both on polyvalent anti-IgM (cross-reactive with monkey) and monoclonal anti-IgM, (non-crossreactive with monkey).

Sera were diluted 1:200 in serum diluent and the monkey serum was diluted 1:100, 1:200 and 1:400.

Serum incubation one hour at 37° C., conjugate incubation one hour at 37° C., and TMB (ready to use): 30 minutes at room temperature. The reaction was stopped with sulphuric acid (0.5M).

Results were interpreted by eye. Three of the four SARS-IgM positive sera (as detected by IF on infected cells) had a higher score than negative control sera. One serum had a score which was also reached by some of the negative controls. The 9 day old monkey sera did not react, but the 12 day old did. Thus, this study shows that with direct conjugation of nucleocapsids the developemnt of an IgM capture method is feasable.

Furthermore, this type of assay can be performed in a number of formats by those trained in the art. The assay can be extended to the detection of IgA and IgG antibodies from humans and animals and can make use of different capture antigens, such as, but not limited to, purified recombinant N protein.

Animal Immunisation

Cynomologous macaque specific antisera for the newly discovered virus were generated by experimental intratrachael installation of cultured virus of Cynomologous macaques. One to two weeks later the animals were bled. The sera were tested for reactivity to SARS virus by indirect IFA as described above; uninfected control cells were used to ensure the specificity of the serum. Other animal species are also suitable for the generation of specific antibody preparations and other antigen preparations may be used.

RNA Isolation

RNA was isolated from the supernatant of infected cell cultures or sucrose gradient fractions using a High Pure RNA Isolation kit according to instructions from the manufacturer (Roche Diagnostics, Almere, The Netherlands). RNA can also be isolated following other procedures known in the field (Current Protocols in Molecular Biology).

RT-PCR

A one-step RT-PCR was performed in 50 μl reactions containing 50 mM Tris.HCl pH 8.5, 50 mM NaCl, 4 mM MgCl₂, 2 mM dithiotreitol, 200 μM each dNTP, 10 units recombinant RNAsin (Promega, Leiden, the Netherlands), 10 units AMV RT (Promega, Leiden, The Netherlands), 5 units Amplitaq Gold DNA polymerase (PE Biosystems, Nieuwerkerk aan de Ijssel, The Netherlands) and 5 μl RNA. Cycling conditions were 45 min. at 42° C. and 7 min. at 95° C. once, 1 min at 95° C., 2 min. at 42° C. and 3 min. at 72° C. repeated 40 times and 10 min. at 72° C. once.

Primers Used for Diagnostic PCR:

For the amplification of the SARS virus' genetic material, we used primers:

SARS fwd2: ggtggaacatcatccggtgat
SARS rev2: agcctgtgttgtagattgcgg

These primers amplify a 149 bp fragment of the polymerase gene (orf 1ab) RT-PCR, gel purification and direct sequencing were performed as described above.

Sequence Analysis

Sequence analyses were performed using a Dyenamic ET terminator sequencing kit (Amersham Pharmacia Biotech, Roosendaal, The Netherlands) and an ABI 373 automatic DNA sequencer (PE Biosystem). All techniques were performed according to the instructions of the manufacturer. PCR fragments were sequenced directly with the same oligonucleotides used for PCR, or the fragments were purified from the gel with Qiaquick Gel Extraction kit (Qiagen, Leusden, The Netherlands) and cloned in pCR2.1 vector (Invitrogen, Groningen, The Netherlands) according to instructions from the manufacturer and subsequently sequenced with M13-specific oligonucleotides.

Detection of Antibodies in Humans, Mammals, Ruminants or Other Animals by ELISA

A recombinant protein derived from the SARS virus is preferred as the antigen. However, purified nucleocapsids may also be used. Antigens suitable for antibody detection include any SARS protein that combines with any SARS-specific antibody of a patient exposed to or infected with SARS virus. Preferred antigens of the invention include those that predominantly engender the immune response in patients exposed to SARS, which therefore, typically are recognised most readily by antibodies of a patient. Particularly preferred antigens include the N, and S proteins of SARS.

Antigens used for immunological techniques can be native antigens or can be modified versions thereof. Well known techniques of molecular biology can be used to alter the amino acid sequence of a SARS antigen to produce modified versions of the antigen that may be used in immunologic techniques.

Methods for cloning genes, for manipulating the genes to and from expression vectors, and for expressing the protein encoded by the gene in a heterologous host are well-known, and these techniques can be used to provide the expression vectors, host cells, and the for expressing cloned genes encoding antigens in a host to produce recombinant antigens for use in diagnostic assays. See for instance: Molecular cloning, A laboratory manual and Current Protocols in Molecular Biology.

A variety of expression systems may be used to produce SARS antigens. For instance, a variety of expression vectors suitable to produce proteins in E. Coli, B. subtilis, yeast, insect cells and mammalian cells have been described, any of which might be used to produce a SARS antigen suitable to detect anti-SARS antibodies in exposed patients.

The baculovirus expression system has the advantage of providing necessary processing of proteins, and is therefor preferred. The system utilizes the polyhedrin promoter to direct expression of SARS antigens. (Matsuura et al. 1987, J. Gen. Virol. 68: 1233-1250).

Antigens produced by recombinant baculo-viruses can be used in a variety of immunological assays to detect anti-SARS antibodies in a patient. It is well established, that recombinant antigens can be used in place of natural virus in practically any immunological assay for detection of virus specific antibodies. The assays include direct and indirect assays, sandwich assays, solid phase assays such as those using plates or beads among others, and liquid phase assays. Assays suitable include those that use primary and secondary antibodies, and those that use antibody binding reagents such as protein A. Moreover, a variety of detection methods can be used in the invention, including colorimetric, fluorescent, phosphorescent, chemiluminescent, luminescent and radioactive methods.

### Example 3

### Animal Models

Macaques

Four Cynomologous Macaques were infected with SARS virus by intratrachaeal installation using Vero-118 cell derived virus.

The monkeys had the following clinical symptoms
Lethargy
One of four monkeys had severe pneumonia
Mild to severe rash in the inguinal region and the axilar region
Watery stools

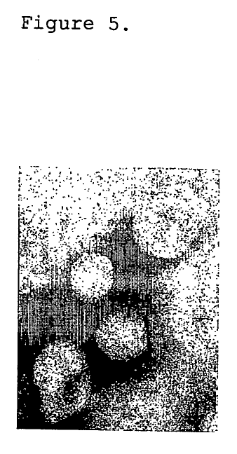

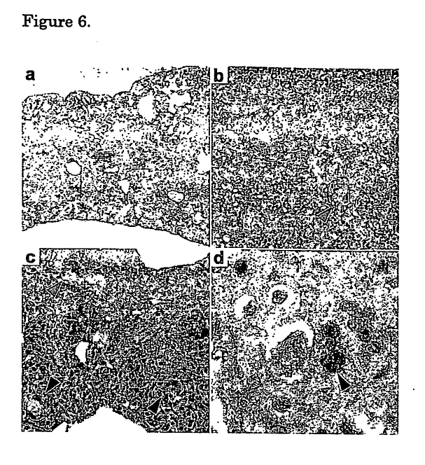

After 10-16 days the monkeys were euthanized. Tissues were examined and the following was found
Alveolae were filled with serum and their architecture were disrupted, consistent with bronchointestitial pneumonia (see FIG. 5 and b)
Multi-cell syncytia in lungs (FIG. 5c)
Multi-cell syncytia in kidneys (FIG. 5d)
Widening of the small intestine

Virus was detected using RT-PCR on tissue samples and by culturing samples followed by electron microscopy from
Lungs
Nasal swabs
Throat swabs
Faeces
Kidneys

The EM results demonstrate that the virus that was recovered from the Cynomologous Macaques had the identical morphology to the virus which was used to infect them.

This demonstrates that Cynomologous Macaques may be used as animal models to tests the efficacy of pharmaceutical preparations for therapeutic or prophylactic purposes

Cats and Ferrets

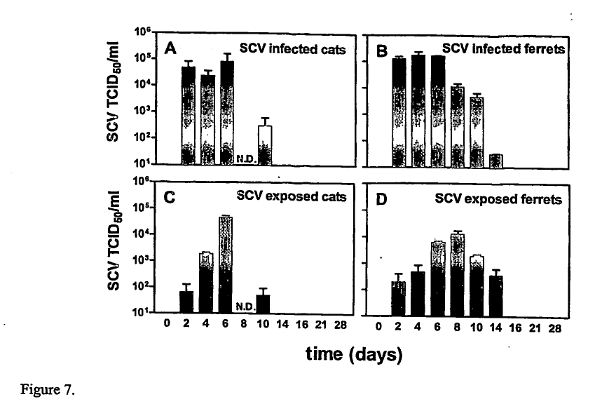

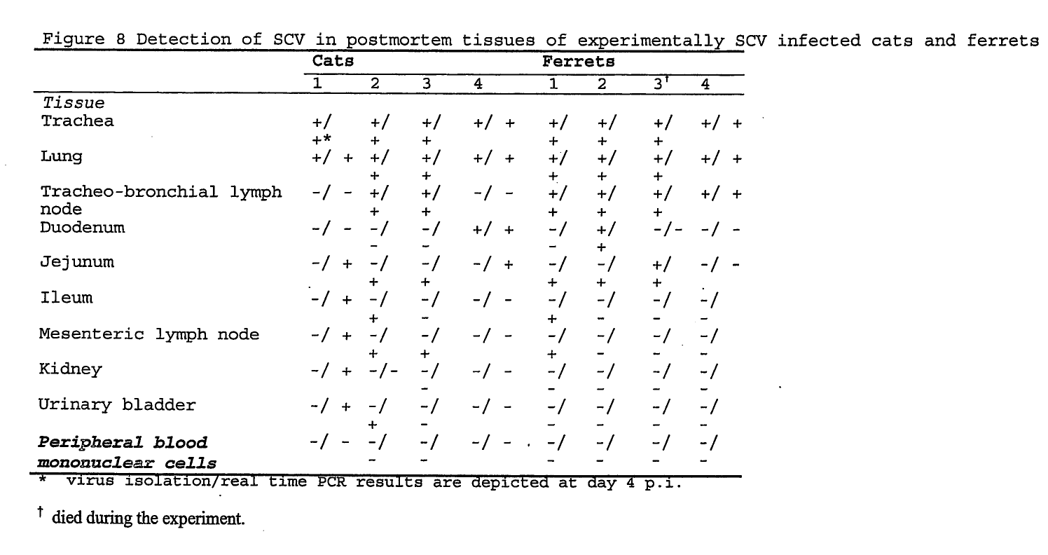

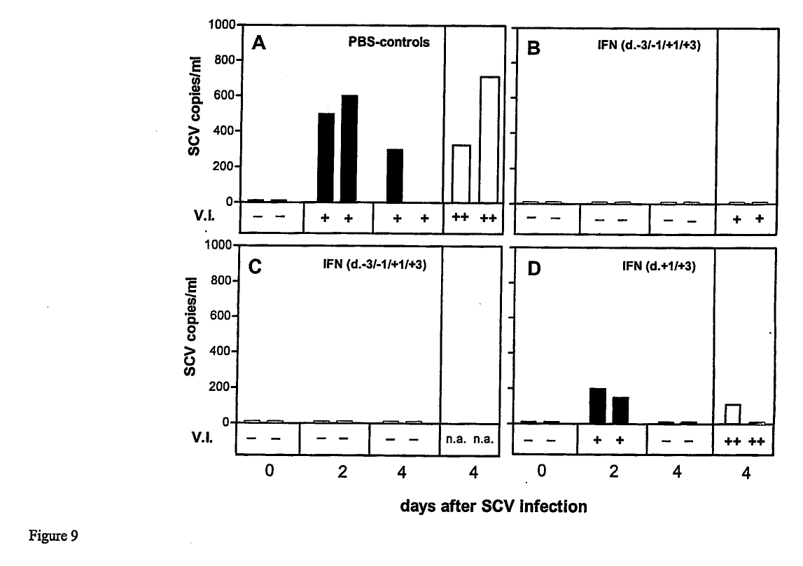

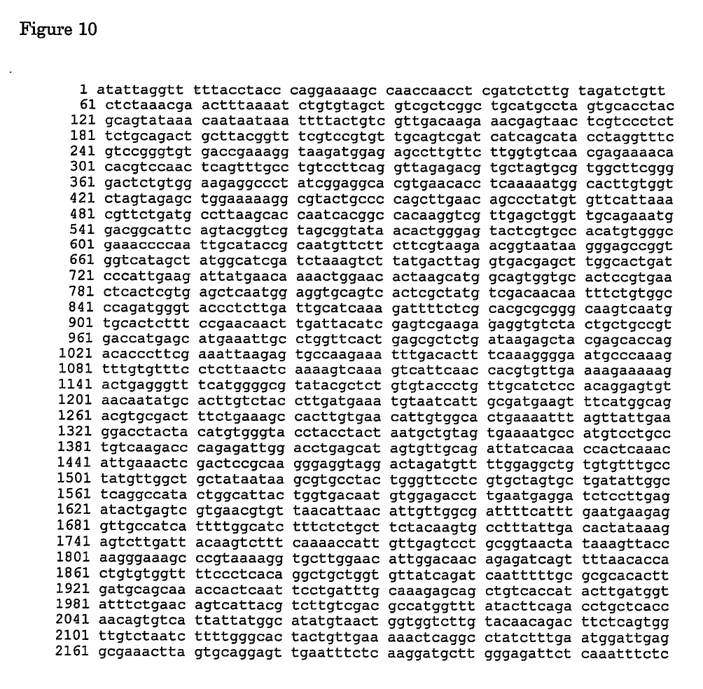

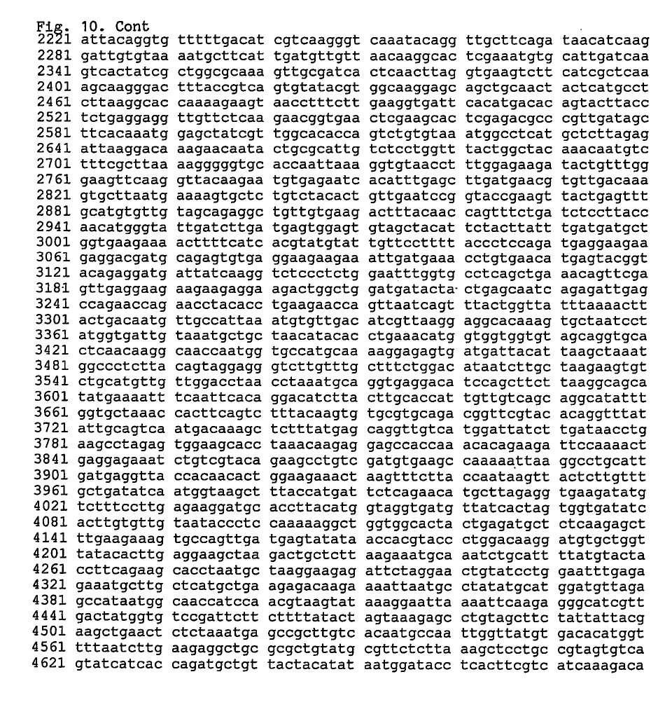

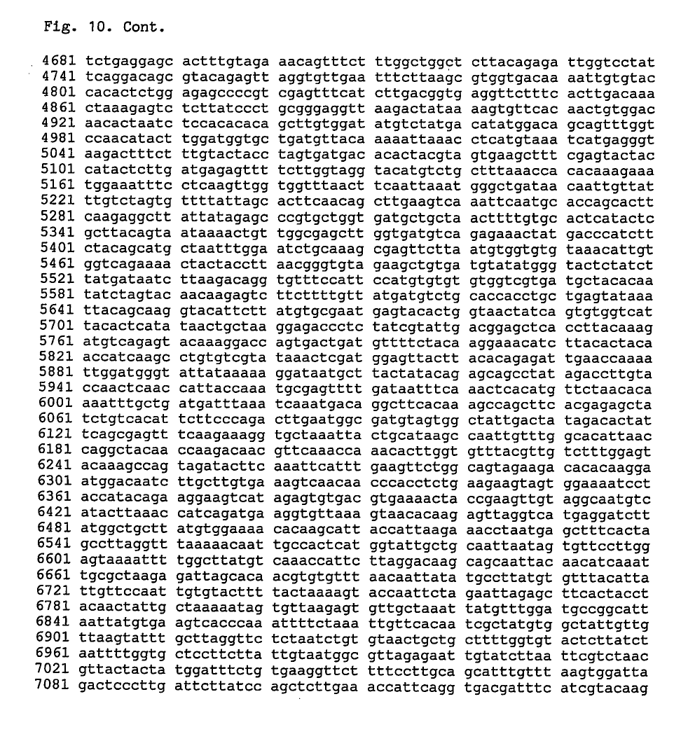

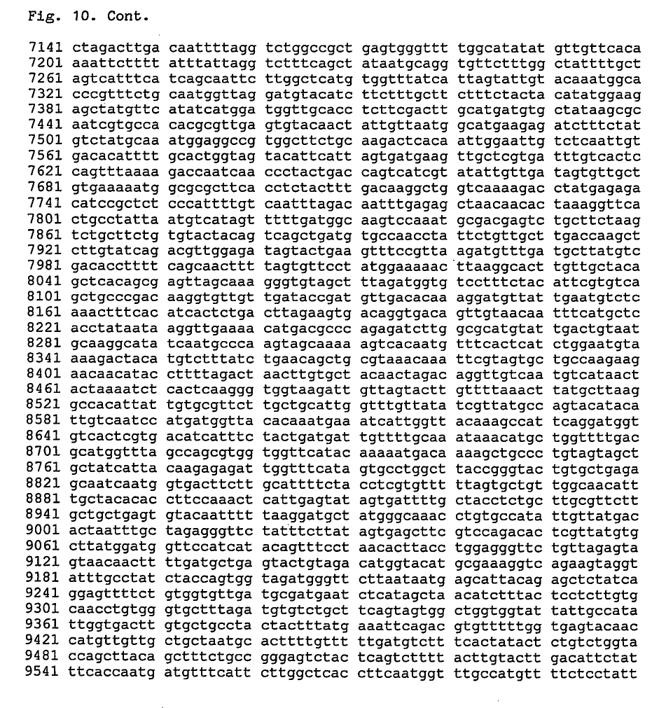

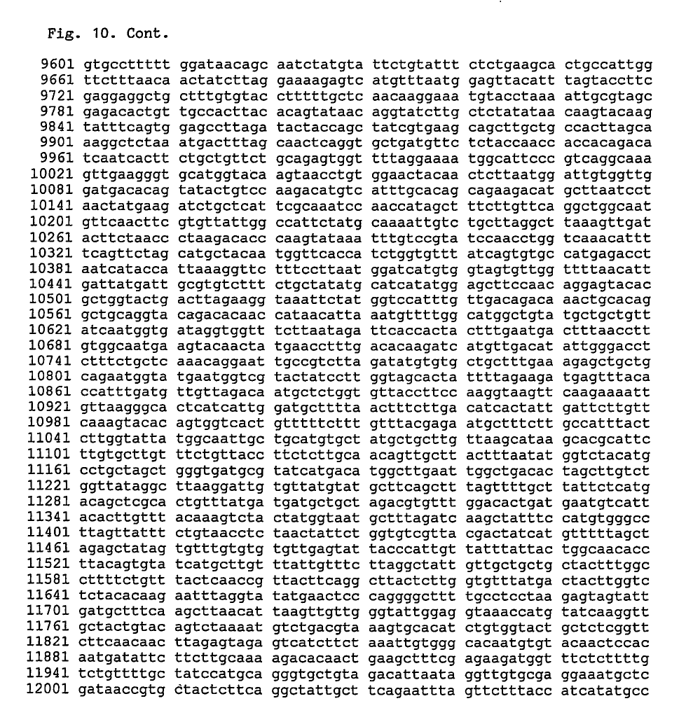

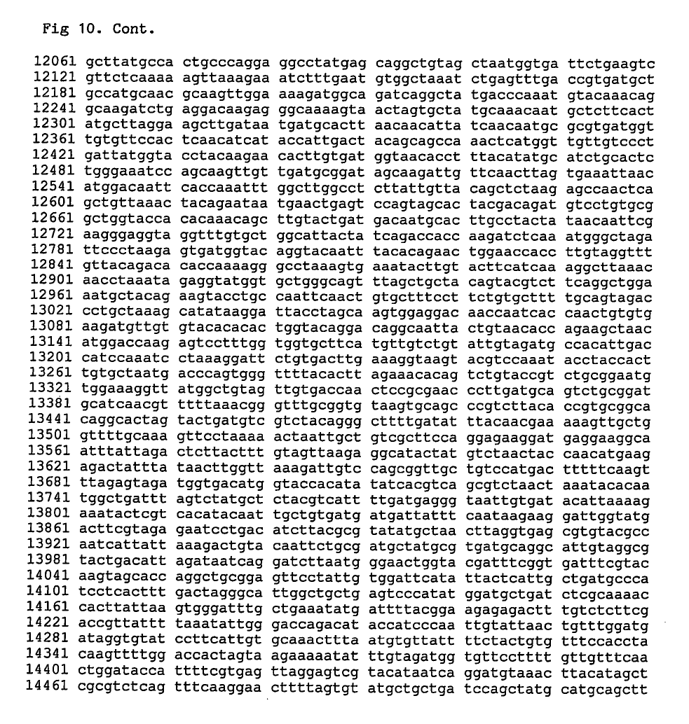

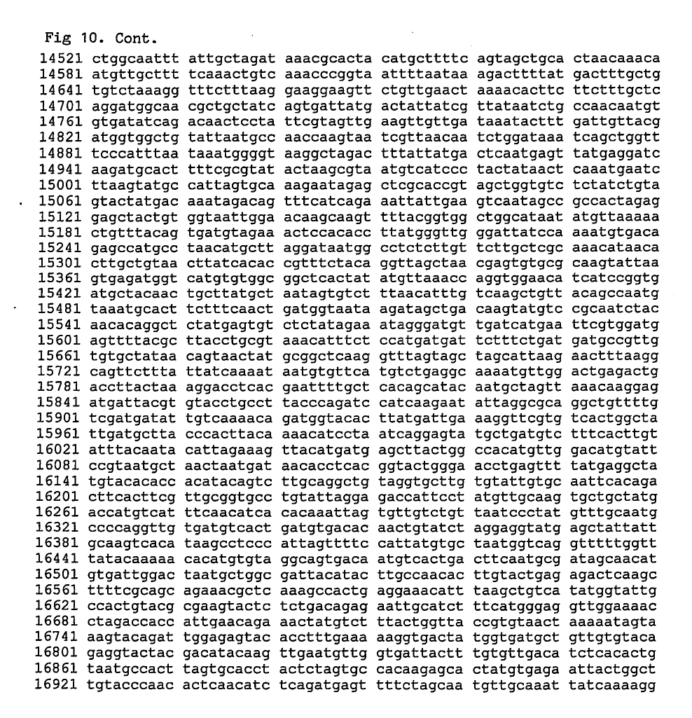

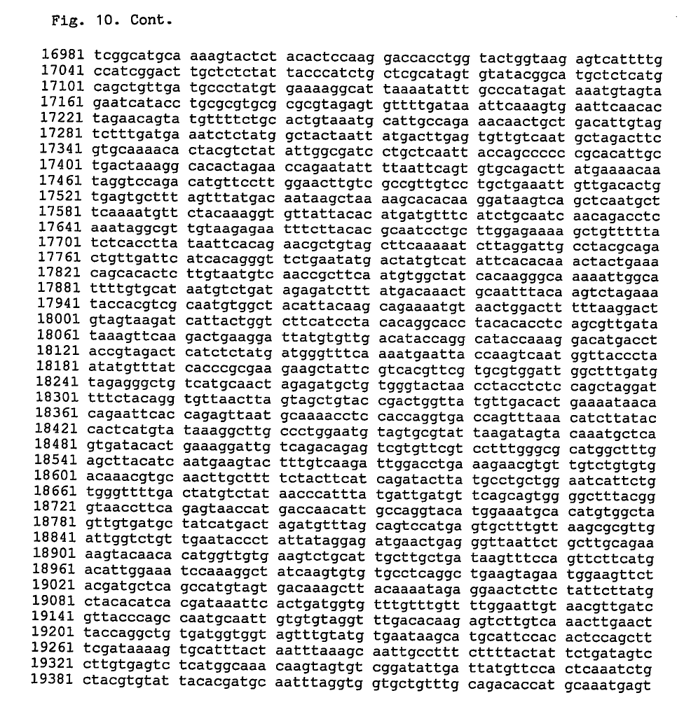

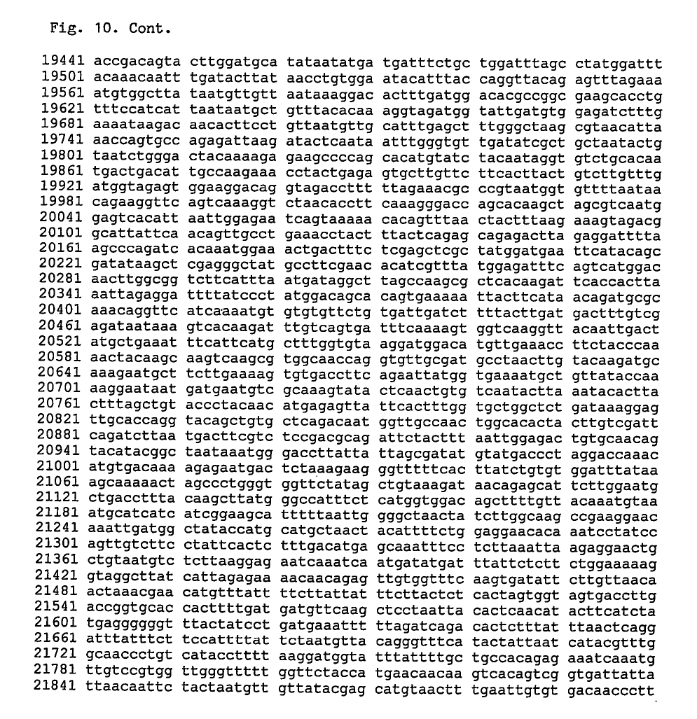

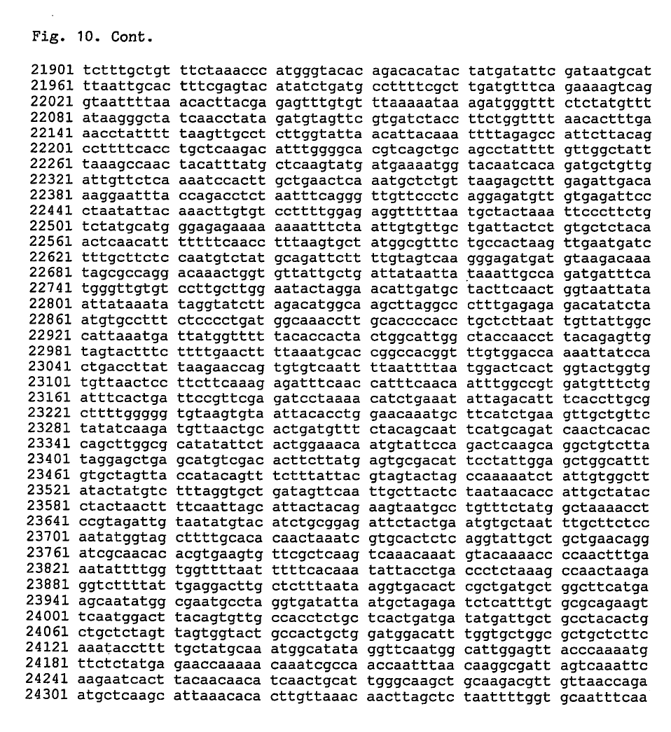

Domestic cats (n=6) and ferrets (n=6) were inoculated intratracheally with 106 median tissue culture infectious dose (TCID50) SCV, obtained from patient 5688 who died from SARS and passaged four times on Vero 118 cells in vitro. Nasal, pharyngeal and rectal swabs were taken on different days post infection (d.p.i.). Four animals of each group were euthanised at 4 d.p.i. and necropsy was performed according to a standard protocol. No clinical signs were observed in SCV-inoculated cats, while three out of six ferrets became lethargic from 2 to 4 d.p.i. and one of these ferrets died at 4 d.p.i. All cats and ferrets (FIG. 7) shed SCV from the pharynx starting at 2 d.p.i. until day 10 and 14, respectively, as determined by RT-PCR. Virus was isolated from all pharyngeal swabs taken on 2-8 d.p.i. and nasal swabs of two cats on 4 and 6 d.p.i. SCV was detected neither in nasal swabs from ferrets nor in rectal swabs of cats or ferrets. Infection of the respiratory tract was evident in all animals tested; SCV could be isolated from their tracheas and lungs (FIG. 8). Quantification of the mean geometric viral titres per ml lung homogenate revealed relatively low SCV titers in the lungs of SCV-inoculated cats (1×103±0.51 TCID50) compared to ferrets (1×106±0.70 TCID50). Histologically, SCV infection was associated with pulmonary lesions similar to those in SCV-infected macaques, except that they were milder, especially in SCV infected cats and syncytia were not found. In the gastro-intestinal and urinary tracts SCV was detected by RT-PCR (FIG. 8). Follow up of the remaining SCV-inoculated animals (n=2 per group) revealed that they all had seroconverted by 28 d.p.i. (neutralising antibody titers 40-320). Two attempts to infect suckling mice through intracerebral inoculation failed.

Non-inoculated cats (FIG. 7c, n=2) and ferrets (FIG. 7d, n=2) housed together with the inoculated cats and ferrets, respectively, became infected with SCV; viral titers gradually increased from day 2 onwards and peaked at 6 to 8 d.p.i. Neither of the cats showed clinical signs but had seroconverted by day 28 (virus neutralising antibody titres of 40 and 160). Both ferrets showed lethargy and conjunctivitis and died on 16 and 21 d.p.i. Based on pathologic examination, the main lesions in these two animals were marked hepatic lipidosis and emaciation. There was no evidence that either of these animals died of SCV-associated pneumonia, although SCV was isolated from postmortem lung specimens of one animal.

In conclusion, domestic cats and ferrets are susceptible to experimental SCV infection and transmission of SCV to non-inoculated animals occurs efficiently. Both species potentially could be used as animal models to test antiviral drugs or vaccine candidates against SARS.

### Example 4

### SARS-Interferon Experiments

In a first experiment four groups of two monkeys were injected.

1. PEG-INTERFERON Treatment

Dose: 3 μg/kg or PBS injected intramuscularly according the following scheme:

| Monkey: | |
|---|---|
| M001 | PBS at days −3, −1, +1 and +3 |
| M002 | PBS at days −3, −1, +1 and +3 |
| M003 | IFN at days −3, −1, +1 and +3 |
| M004 | IFN at days −3, −1, +1 and +3 |
| M005 | PBS at d.−3 and −1 and IFN at d.+1 and +3 |
| M006 | PBS at d. −3 and −1 and IFN at d. +1 and +3 |
| M007 | IFN at days −3, −1, +1 and +3 |
| M008 | IFN at days −3, −1, +1 and +3 |

2. Infection

SARS coronavirus infection of all monkeys on day 0

Dose: 10⁶ TCID 50 in 5 ml PBS
4 ml intra-tracheal
1 ml intranasal
0.5 ml on each of the eyes

3. Sampling
a. Nose throat and rectum swabs taken on days 0, 2 and 4 and were put in 1 ml transport medium.
b. Monkeys were euthanised on day 4 and samples of lung, tracheal bronchial lymph node and trachea were harvested

Virus was cultured and titrated on Vero-118 cells, and these were scored for cytopathic affects

Virus titration using the three different swabs taken on days 0, 2 and 4 after infection (nose, throat and rectum) and isolation of virus from the lungs, tracheal bronchial lymph node and trachea at day 4 after infection demonstrated that the two control monkeys (M001 and M002) were successfully infected (table 1).

TABLE 1
SARS-associated coronavirus excretion by cynomolgus
macaques treated with pegylated interferon.

| | specimen* | | | | | |
|---|---|---|---|---|---|---|
| | Pharyngeal swab | | | Tr. Br lymph node | Trachea | Lung |
| Animal no. | 0 | 2 | 4 | 4 | 4 | 4 |
| M001 | − | + | + | + | + | + |
| M002 | − | + | + | + | + | ++ |
| M003 | − | − | − | − | − | + |
| M004 | − | − | − | + | − | + |
| M005 | − | + | − | − | + | ++ |
| M006 | − | + | − | + | + | ++ |
| M007 | − | − | − | n.a. | n.a. | n.a. |
| M008 | − | − | − | n.a. | n.a. | n.a. |

*day post infection

1. Control Animals (M001, 002)
Pharyngeal swabs on days 2 and 4 were all positive
Animal M001 also was found positive with respect to isolation of SARS coronavirus from the nasal swab (day 2 and 4).
No rectal swabs were positive
Tissue specimen from the lungs, trachea and trachea bronchial lymph node from both control animals (M001 and M002) were positive at day 4 when the animals were sacrificed. The lung tissue homogenate contained virus at a high titer because the Vero cultures were found positive rapidly after inoculation.

2. Prophylactically Treated Animals (M003, 004, 007 & 008)
negative with respect to the virus isolation test on pharyngeal swabs taken at day 0, 2 and 4 after infection (table 1).
No nasal swab was found positive in these animals.
Only one rectal swab of animal M004 at day 4 was scored positive (which has to be confirmed in the PCR assay because these cultures showed much bacterial contamination (cultures of rectal swabs)
No virus isolated from trachea of M003 and 004
Virus isolated from tracheal bronchial lymph node of M004 but not M003
Virus isolated from lungs of M003 and 004, but are at lower titre than controls as it took longer for CPE to be observed in Vero-118 cells inoculated with samples from the lungs (confirmed by PCR—FIG. A below)

3. Therapeutically Treated Animals (M005 and 006)

SARS Coronavirus
isolated from pharyngeal swabs taken at day 2 after infection
not isolated from the pharyngeal swabs taken at day 4 after infection.
isolated from more tissue samples and at higher titers from animal M005 and M006, than from animal M003 and M004 (quantitation confirmed by PCR)

Pathological examination of lung section stained by HE confirmed the low level infection of the lungs of animal M003.

In a second experiment treatment of Cynomolgus macaques was preceded by an in vitro dose-finding experiemnt on Vero cells.

In vitro Study

Wells containing Vero cells were treated in triplicate with pegylated recombinant IFN-α (PEG-Intron, Shering Corp) for 16 h and infected with 100 TCID₅₀ per well of SCV, obtained from patient 5688, who died of SARS. After 16 h the supernatant was removed and cells were fixed by 10% neutral-buffered formalin and 70% ethanol (10 min RT). SCV antigen positive cells were visualised by immunohistochemistry, as described under histology. The number of SCV-infected cells per well was summarized as mean±s.d.

Macaque Studies.

Three groups of cynomolgus macaques were infected intratracheally with 1×10⁶ TCID₅₀ SCV suspended in 5 ml of phosphate buffered saline (PBS). One (the control group, n=4) was injected intramuscularly with PBS and two (the prophylactic group, n=6; the post-exposure group, n=4) with pegylated IFN-α at a dose of 3 μg/kg. The prophylactic group was injected with pegylated IFN-αat days −3, −1, 1 and 3 after SCV infection and the post-exposure group at days 1 and 3 after SCV infection. Four macaques from each group were euthanised at day 4 after infection. Approval for the animal experiments had been obtained from the Institutional Animal Welfare Committee. At days −3, −2, −1, 0, +2 and +4, we anaesthetised the macaques with ketamine and collected 10 ml blood from inguinal veins and took pharyngeal swabs, which were placed in 1 ml transport medium (Fouchier, R. A. et al., J. Clin. Microbiol. 38, 4096-5001). Pharyngeal swabs were frozen at −70° C. until RT-PCR analysis. Pegylated IFN-α levels were determined using an ELISA (Bender MedSystems Diagnostics) using PEG-Intron as a standard and neopterin levels were determined as described by van Gool et al. (Psychiatry Res. 119, 125-132, 2003). Necropsies were done according to a standard protocol; one lung of each monkey was inflated with 10% neutral-buffered formalin by intrabronchial intubation and suspended in 10% neutral-buffered formalin overnight. Samples were collected in a standard manner (one from the cranial part of the lung, one from the medial and two from the caudal part), embedded in paraffin, cut at 5 μm and used for immunohistochemistry (see below) or stained with haematoxylin and eosin (HE). For semiquantitative assessment of SCV-infection-associated inflammation in the lung, each HE-stained section was examined for inflammatory foci by light microscopy using a 10× objective. Each focus was scored for size (1: smaller than or equal to area of 10× objective, 2: larger than area of 10× objective and smaller than or equal to area of 2.5× objective, 3: larger than area of 2.5× objective) and severity of inflammation (1: mild, 2: moderate, 3: marked). The cumulative scores for the inflammatory foci provided the total score per animal. Sections were examined without knowledge of the identity of the macaques. The lung sections of one monkey in the post exposure group were not assessed because of the presence of inflammation from pre-mortem aspiration of food remains. Lung samples from a control group macaque were used for transmission electron microscopy as described by Kuiken, T. et al. (Lancet 362, 263-270, 2003).

Three lung tissue samples taken from the other lung (one from the cranial part of the lung, one from the medial part, and one from caudal part) were homogenised in 2 ml transport medium using Polytron PT2100 tissue grinders (Kinematica). After low speed centrifugation, the homogenates were frozen at −70° C. until inoculation on Vero 118 cell cultures in 10-fold serial dilutions. The identity of the isolated virus was confirmed as SCV by RT-PCR of supernatant.

Immunohistochemistry

The same formalin-fixed paraffin-embedded lung samples as used for histology—one from the cranial part of the lung, one from the medial part, and two from caudal part—were cut at 5 μm, and stained for SCV antigen using a biotinylated purified human IgG from a convalescent SARS patient, negative control biotinylated purified human IgG, or the dilution buffer, as described by Kuiken et al. (supra). Twenty-five arbitrarily chosen 20× objective fields of lung parenchyma in each lung section were examined by light microscopy for the presence of SCV antigen expression, without knowledge of the identity of the macaques. The cumulative scores for each animal were expressed as number of positive fields per 100 fields (%). Selected lung sections from macaques in the control group were stained with anti-cytokeratin monoclonal antibody AB1/AE3 (Neomarkers) for identification of epithelial cells, according to standard immunohistochemical procedures.

SCV RT-PCR

An RT-PCR with primers and probe specific for the nucleoprotein (NP) gene of SCV was used to quantificate SCV in swabs as described by Kuiken et al. (supra). Serial dilutions of the SCV stock were used as a standard and the results were expressed as SCV eq/ml swab medium.

Results

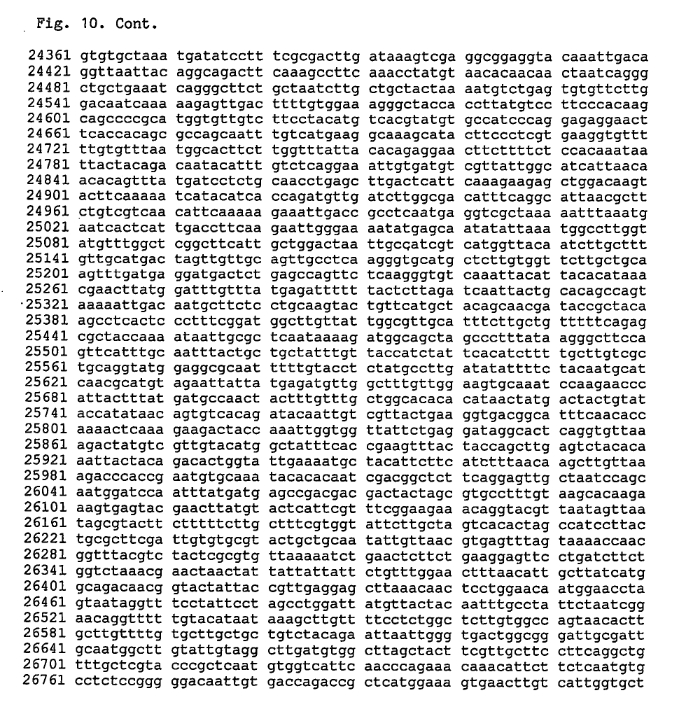

The dose finding study (3 separate experiments) on Vero cells showed a dose-dependent effect on the numbers of SCV infected cells per well. A significant effect was already observed at a dose of 1 ng/ml drug, while a dramatic reduction in the number of infected cells was observed at doses higher than 1 ng/ml (FIG. 11a).

The control macaques showed multifocal acute DAD (diffuse alveolar damage), characterized by flooding of alveoli with protein-rich oedema fluid mixed with neutrophils and rare syncytia, extensive loss of alveolar and bronchiolar epithelium and occasional type 2 pneumocyte hyperplasia. As indicated by immunohistochemistry, there was extensiveSCV antigen expression of squamous cells lining the alveolar walls. They were indicated as type 1 pneumocytes by their location, morphology, and expression of keratin in serial sections. By transmission electron microscopy, coronavirus-like particles measuring about 70 nm in diameter with typical internal nucleocapsid-like structure were found in alveolar cells. These cells were identified as type 1 pneumocytes because they lined the alveolar lumen, were closely apposed to the basement membrane, were squamous, contained abundant pinocytotic vesicles, and—in contrast to type 2 pneumocytes—had neither lamellar bodies nor microvilli. As found previously in experimentally infected macaques at 6 d.p.i., less extensive SCV antigen expression also was detected in hyperplastic type 2 pneumocytes within inflammatory foci. The combination of these histopathologic and immunohistochemical findings show that type 1 pneumocytes are the main target of SCV in early infection, and are associated with DAD.

High plasma levels of pegylated IFN-α after intramuscular injection into a group of six macaques (prophylactic group) were attained 1 day after injection (FIG. 11b), similar to peak levels found in patients after subcutaneous injection with 3 μg/kg pegylated IFN-α (Bukowski, R. M. et al., Cancer 95, 389-396, 2002). Because IFN-α is known to activate macrophages (van Gool et al., supra), plasma levels of neopterin following pegylated IFN-α treatment were measured as a measure of macrophage activation. Neopterin levels were increased in all animals (FIG. 11c), confirming the biological availability of pegylated IFN-α in the treated macaques.

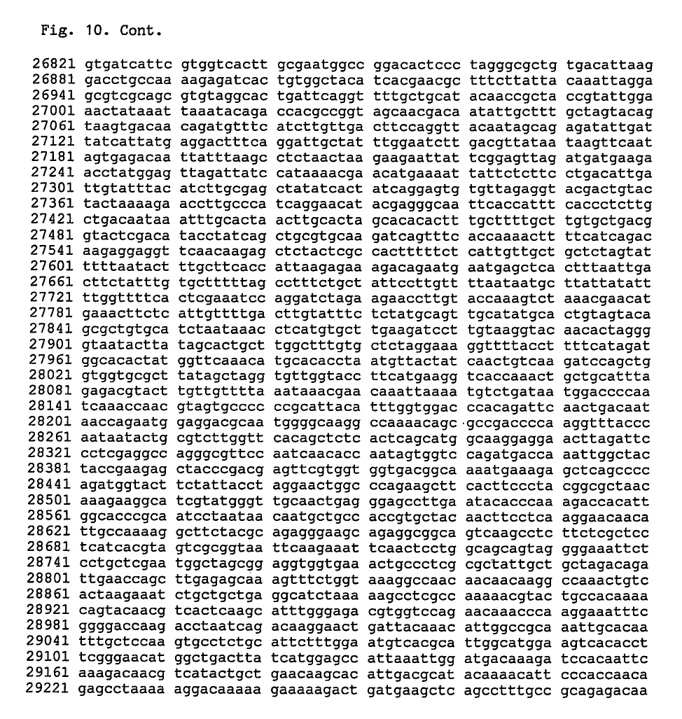

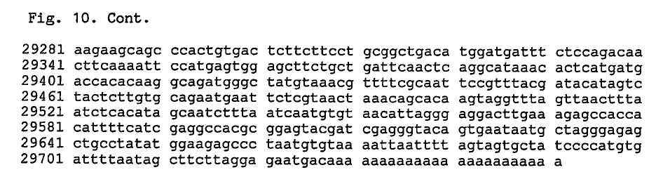

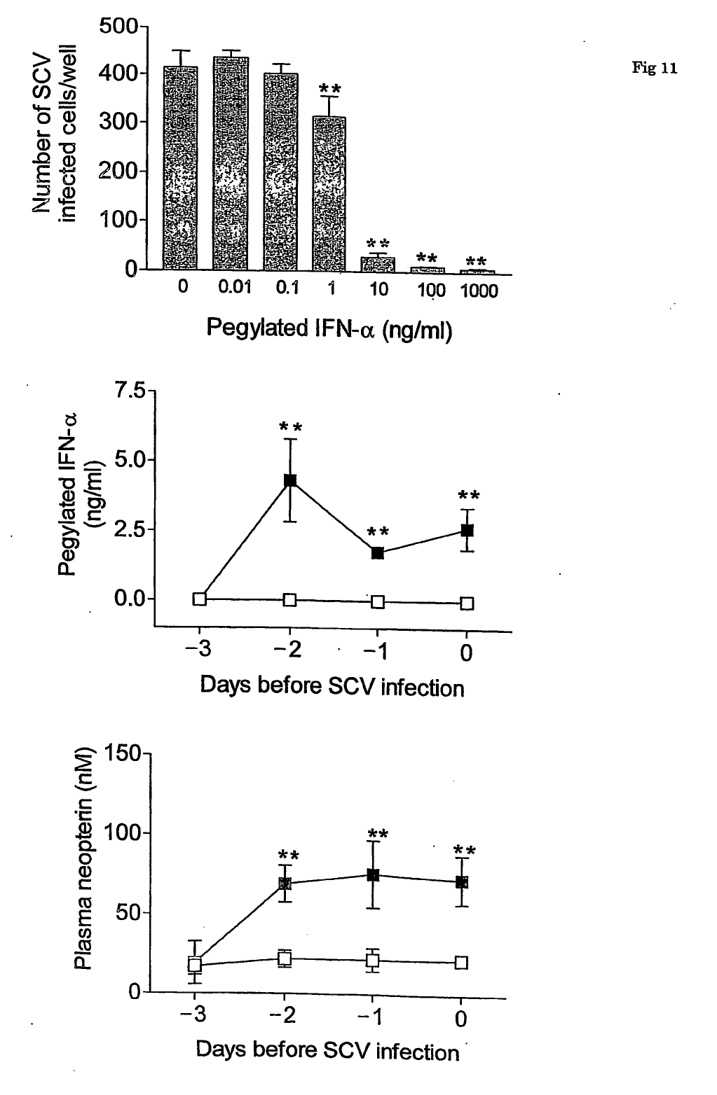

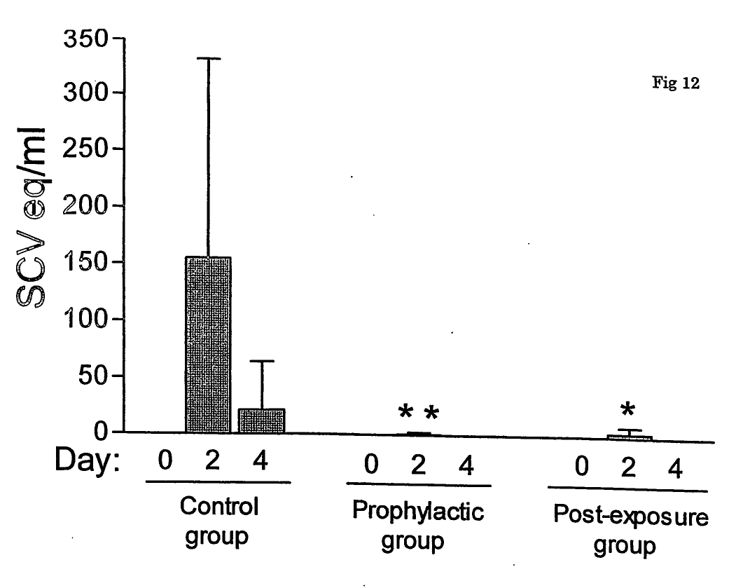

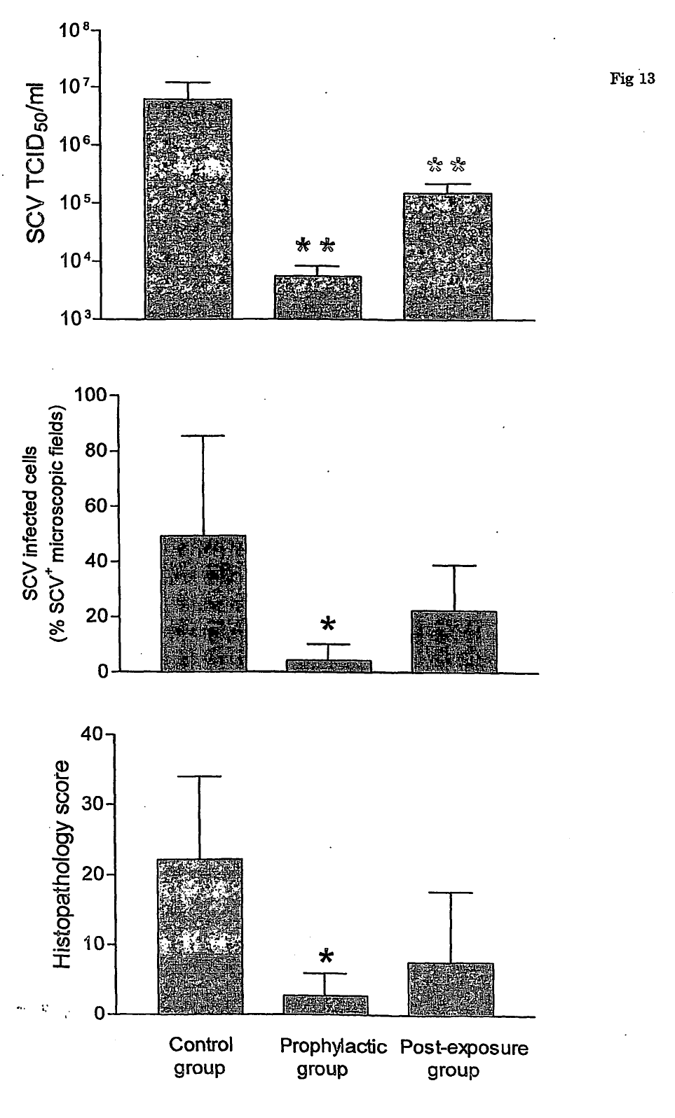

To evaluate the prophylactic use of pegylated IFN-α, we experimentally infected the macaques in the prophylactic group with SCV at 3 days after the start of pegylated IFN-α treatment, and compared virological and pathological parameters with a control group of four macaques treated with PBS instead. We limited our investigation to the pharyngeal swabs and the lung because an earlier study did not provide evidence of extensive viral replication in other organs (Kuiper et al., supra). We found that all parameters were significantly reduced in the prophylactic group compared to the control group. By virology, virus excretion from the pharynx was abrogated (FIG. 12), and the virus titre in the lungs at 4 d.p.i. was significantly reduced (FIG. 13a). By immunohistochemistry, the expression of SCV in type 1 pneumocytes was 90% reduced (FIG. 13b). By pathology, the extent and severity of DAD was 80% reduced (FIG. 13c). These data demonstrate that prophylactic use of pegylated IFN-α substantially, although not completely, protects type 1 pneumocytes of experimentally infected macaques from SCV infection, with abrogation of virus excretion and reduced severity of pulmonary lesions.

To test the efficacy of pegylated IFN-α as an antiviral agent post-exposure, we injected pegylated IFN-α intramuscularly into a post-exposure group of four macaques 1 and 3 days after experimental SCV infection, and evaluated them in the same way as the prophylactic group. Excretion of SCV from the pharynx was found only on 2 d.p.i. at a significantly reduced level compared to the control group (FIG. 12). Moreover, the virus titre in the lungs at 4 d.p.i. was significantly decreased, whereas the remaining parameters were less reduced (FIG. 13a-c). These results show that use of pegylated IFN-α one day post-exposure protects type 1 pneumocytes of experimentally infected macaques from SCV infection but is less effective than prophylactic use.

In this study, we have shown that type 1 pneumocytes are the main target cell for SCV infection of cynomolgus macaques early in the disease, and that pegylated IFN-α protects type 1 pneumocytes from SCV infection. The first point—type 1 pneumocytes as the primary target cell—is evident from the extensive presence of SCV in type 1 pneumocytes at 4 d.p.i). The temporal sequence of lung lesions that emerges when the pathological studies in humans and macaques are viewed together is: viral infection and subsequent loss of type 1 pneumocytes; acute DAD, characterized by flooding of alveolar lumina with highly proteinaceous oedema fluid; chronic DAD, characterized by type 2 pneumocyte hyperplasia; and, in severe cases, extensive pulmonary fibrosis. This sequence of events corresponds to the stereotypic alveolar reaction to acute lung injury from a variety of causes (Ware, L. B. and Matthay, M. A., N. Eng. J. Med. 342, 1334-1349, 2000).

The second point—that pegylated IFN-α protects type 1 pneumocytes from SCV infection—is based on the beneficial effect of pegylated IFN-α therapy initiated 3 days before SGV inoculation of macaques. In these macaques, SCV infection of type 1 pneumocytes and severity of lung lesions were significantly reduced (FIG. 13), and viral excretion was abrogated (FIG. 12). Pegylated IFN-α treatment thus has an important effect on the outcome of SARS. Therefore, reduction of the viral load by pegylated IFN-α therapy at an early stage of SCV infection helps to prevent serious or fatal outcome of SARS associated with pulmonary fibrosis. In addition to potential disease mitigation, reduced viral excretion through pegylated IFN-α therapy also has an epidemiological effect by reducing the spread of SCV in the human population. Whether the mechanism of pegylated IFN-α protection is by direct antiviral activity or immunostimulatory effects remains to be determined.

The time interval during which effective post-exposure treatment with pegylated IFN-α can be initiated may be longer in humans than in the experimentally infected macaques. This is because the peak of SCV infection in the lungs is at about 16 d.p.i. in humans—based on an average incubation period of 6 days (Booth, C. M. et al, JAMA 289, 2801-2809, 2003) and a peak in viral excretion at 10 days after onset of symptoms (Peiris, J. S. M. et al, Lancet 361, 1767-1772, 2003)—compared to 2 d.p.i. in these macaques (FIG. 12).

In conclusion, these studies show that type 1 pneumocytes are the main target cell for SCV infection of macaques early in the disease, and that pegylated IFN-α, a commercially available antiviral drug, protects these cells from SCV infection. Prophylactic or early post-exposure treatment with pegylated IFN-α will help to reduce the impact of SCV infection on healthcare workers and others possibly exposed to SCV and to limit the spread of the virus in the human population.

1-42. (canceled)

43. An isolated essentially mammalian positive-sense single stranded RNA virus (SARS) comprising one or more of the sequences of FIG. 2.

44. An isolated positive-sense single stranded RNA virus (SARS) belonging to the Coronaviruses and identifiable as phylogenetically corresponding thereto by determining a nucleic acid sequence of said virus and testing it in phylogenetic tree analyses wherein maximum likelihood trees are generated using 100 bootstraps and 3 jumbles and finding it to be more closely phylogenetically corresponding to a virus isolate having the sequences as depicted in FIG. 2 than it is corresponding to a virus isolate of BoCo (bovine coronavirus), MHV (murine hepatitis virus), AIBV (avian infectious bronchitis virus), PEDV (porcine epidemic diarrhea virus), TGEV (transmissible gastroenteritis virus) or 229E (human coronavirus 229E).

45. A virus according to claims 43 or 44 wherein said nucleic acid sequence comprises an open reading frame (ORF) encoding a viral protein of said virus, preferably selected from the group of ORFs encoding the viral replicase, nuclear capsid protein and the spike protein.

46. A virus according to claims 43-45 isolatable from a human with atypical pneumonia.

47. An isolated or recombinant nucleic acid or SARS virus-specific functional fragment thereof obtainable from a virus according to anyone of claims 43 to 46.

48. A vector comprising a nucleic acid according to claim 47.

49. A host cell comprising a nucleic acid according to claim 47 or a vector according to claim 48.

50. An isolated or recombinant proteinaceous molecule or SARS virus-specific functional fragment thereof encoded by a nucleic acid according to claim 47.

51. An antigen comprising a proteinaceous molecule or SARS virus-specific functional fragment thereof according to claim 50.

52. An antibody specifically directed against an antigen according to claim 51.

53. A method for identifying a viral isolate as a SARS virus comprising reacting said viral isolate or a component thereof with a nucleic acid according to claim 47 and/or with an antibody according to claim 52.

54. A method for virologically diagnosing a SARS infection of a mammal comprising determining in a sample of said mammal the presence of a viral isolate or component thereof by reacting said sample with a nucleic acid according to claim 47 or an antibody according to claim 52 or determining in a sample of said mammal the presence of an antibody specifically directed against a SARS virus or component thereof by reacting said sample with a proteinaceous molecule or fragment thereof according to claim 50 or an antigen according to claim 51.

55. A diagnostic kit for diagnosing a SARS infection comprising a virus according to anyone of claims 43 to 46, a nucleic acid according to claim 47, a proteinaceous molecule or fragment thereof according to claim 50, an antigen according to claim 51 and/or an antibody according to claim 52.

56. Use of a virus according to any one claims 43 to 46, a nucleic acid according to claim 47, a vector according to claim 48, a host cell according to claim 49, a proteinaceous molecule or fragment thereof according to claim 50, an antigen according to claim 51, or an antibody according to claim 52 for the production of a pharmaceutical composition, preferably for the production of a pharmaceutical composition for the treatment or prevention of a SARS virus infection or for the production of a pharmaceutical composition for the treatment or prevention of atypical pneumonia.

57. A pharmaceutical composition comprising a virus according to any one of claims 43 to 46, a nucleic acid according to claim 47, a vector according to claim 48, a host cell according to claim 49, a proteinaceous molecule or fragment thereof according to claim 50, an antigen according to claim 51, or an antibody according to claim 52.

58. A method for the treatment or prevention of a SARS virus infection or for the treatment or prevention of atypical pneumonia comprising providing an individual with a pharmaceutical composition according to claim 57.

59. A viral replicase encoded by an RNA sequence comprising the sequences EMC-1, EMC-2, EMC-3, EMC-4, EMC-5, EMC-6, EMC-7, EMC-13 and/or EMC-14, or homologues thereof as depicted in FIG. 2.

60. A viral spike protein comprising the amino acid depicted as translation 2 with sequence EMC-7 and translation 1 of RDG 1 as depicted in FIG. 2, or a homologue thereof.

61. A viral nuclear capsid protein encoded by an RNA sequence comprising the sequence EMC-8 as depicted in FIG. 2 or a homologue thereof.

62. A viral protein encoded by an RNA sequence comprising the sequence EMC-9, EMC-11 and/or EMC-12 as depicted in FIG. 2.

63. A nucleic acid sequence which comprises one or more of the sequences EMC-1 to EMC-13 as depicted in FIG. 13 or a nucleic acid sequence which can hybridise with any of these sequences under stringent conditions.

64. Use of interferon for the preparation of a medicament for the treatment or prevention of a coronavirus associated disease, preferably wherein said interferon is interferon alpha, more preferably wherein said interferon alpha is interferon-alpha 2a or interferon-alpha 2b, and preferably wherein said coronavirus associated disease is a disease of animals, preferably vertebrates, more preferably birds or mammals, especially humans, ape or rodent, more preferably wherein said disease is a respiratory disease and/or gastroenteritis, most preferably wherein said animal is human.

65. Use according to claim 64, wherein said interferon is pegylated.

66. Use according to any of claims 64-65 wherein said coronavirus associated disease is a disease caused by HcoV-NL, the feline infectious peritonitis virus (FIPV) or hemagglutinating encephalomyelitis virus (HEV) of swine or avian infectious bronchitis virus (IBV) or mouse hepatitis virus (MHV), preferably wherein said coronavirus associated disease is a disease caused by a SARS coronavirus (SARS-CoV), more preferably wherein said SARS virus is a positive-sense single stranded RNA virus (SARS coronavirus) comprising one or more of the sequences of FIG. 2, most preferably wherein said SARS virus is a positive-sense single stranded RNA virus (SARS coronavirus) corresponding to GenBank accession no. AY274119 or AY278741. or AY338175 or AY338174 or AY322199 or AY 322198 or AY322197 or AH013000 or AY322208 or AY322207 or AY 322206 or AY322205 or AH012999.

67. A method for the treatment or prevention of a coronavirus associated disease in an animal, preferably a vertebrate, more preferably a bird or mammal, especially human, ape or rodent, infected with a coronavirus, said method comprising administrating interferon, to said animal, preferably a vertebrate, more preferably a bird or mammal, especially human, ape or rodent, along with a pharmaceutically acceptable carrier, preferably wherein said interferon is administered together with a vaccine, antibody and/or antiviral agent, more preferably wherein said vaccine, antibody and/or anti-viral agent is selected from the group consisting of whole inactivated virus vaccines, attenuated vaccines, sub-unit vaccines, recombinant vaccines, antibody for passive immunization, nucleoside analogs such as ribavirin, RNA-dependent RNA polymerase inhibitors, protease inhibitors.
